SEQUENCE LISTING (152 sequences)

SEQ ID NO: 1 - DNA, 27 bp, unidentified
  source 1..27: mol_type = genomic DNA
agctgggacggcaaaggcgctcacttc

SEQ ID NO: 2 - DNA, 27 bp, unidentified
  source 1..27: mol_type = genomic DNA
cgcaaaacctgggctggcaactgggag

SEQ ID NO: 3 - DNA, 27 bp, unidentified
  source 1..27: mol_type = genomic DNA
acctgggacggcaaaggcgctcacttc

SEQ ID NO: 4 - DNA, 27 bp, unidentified
  source 1..27: mol_type = genomic DNA
agcatgtggagcgaccctatggctcac

SEQ ID NO: 5 - DNA, 21 bp, unidentified
  source 1..21: mol_type = genomic DNA
cctccttggctggctgacatc

SEQ ID NO: 6 - DNA, 27 bp, unidentified
  source 1..27: mol_type = genomic DNA
acctgggacggcaaaggcgctcactac

SEQ ID NO: 7 - DNA, 27 bp, unidentified
  source 1..27: mol_type = genomic DNA
agctgggacggcaaaggcgctcactac

SEQ ID NO: 8 - DNA, 27 bp, unidentified
  source 1..27: mol_type = genomic DNA
gtgtgggtgaccggcagcgctcacgac

SEQ ID NO: 9 - DNA, 27 bp, unidentified
  source 1..27: mol_type = genomic DNA
aaatgggacggccgcggcatgcacaaa

SEQ ID NO: 10 - DNA, 27 bp, unidentified
  source 1..27: mol_type = genomic DNA
agctgggacggcaaaggccctcacttc

SEQ ID NO: 11 - DNA, 27 bp, unidentified
  source 1..27: mol_type = genomic DNA
acctgggacggcaaaggccctcacttc

SEQ ID NO: 12 - DNA, 27 bp, unidentified
  source 1..27: mol_type = genomic DNA
aaatgggacggccgcggcatgcacaac

SEQ ID NO: 13 - DNA, 21 bp, unidentified
  source 1..21: mol_type = genomic DNA
gctccttggctggagggcctg

SEQ ID NO: 14 - DNA, 27 bp, unidentified
  source 1..27: mol_type = genomic DNA
agcatctggagcgaccctatggctcac

SEQ ID NO: 15 - DNA, 21 bp, unidentified
  source 1..21: mol_type = genomic DNA
gctccttggctgatggacctg

SEQ ID NO: 16 - DNA, 21 bp, unidentified
  source 1..21: mol_type = genomic DNA
cctgcttggctggaggacctg

SEQ ID NO: 17 - DNA, 21 bp, unidentified
  source 1..21: mol_type = genomic DNA
gaggactggcactggcctctg

SEQ ID NO: 18 - DNA, 30 bp, unidentified
  source 1..30: mol_type = genomic DNA
atctgggacaacgaccgcaaaatgcaccac

SEQ ID NO: 19 - DNA, 27 bp, unidentified
  source 1..27: mol_type = genomic DNA
agctgggacggcaaaggccctcactac

SEQ ID NO: 20 - DNA, 27 bp, unidentified
  source 1..27: mol_type = genomic DNA
acctgggacggcaaaggccctcactac

SEQ ID NO: 21 - DNA, 21 bp, unidentified
  source 1..21: mol_type = genomic DNA
cctccttggctggtgggcctg

SEQ ID NO: 22 - DNA, 27 bp, unidentified
  source 1..27: mol_type = genomic DNA
ttcctgaacagcgagatgctgcacgtg

SEQ ID NO: 23 - DNA, 27 bp, unidentified
  source 1..27: mol_type = genomic DNA
cgcaaaacctgagctggcaactgggag

SEQ ID NO: 24 - DNA, 33 bp, unidentified
  source 1..33: mol_type = genomic DNA
gtgcgctacggcgtgctgaacgcttacacccgc

SEQ ID NO: 25 - DNA, 33 bp, unidentified
  source 1..33: mol_type = genomic DNA
atggacttctggcctatgagccagcagctggct

SEQ ID NO: 26 - DNA, 27 bp, unidentified
  source 1..27: mol_type = genomic DNA
aaccctttcttccctggcttcgacctg

SEQ ID NO: 27 - DNA, 21 bp, unidentified
  source 1..21: mol_type = genomic DNA
gctccttggctgcaggacttc

SEQ ID NO: 28 - DNA, 27 bp, unidentified
  source 1..27: mol_type = genomic DNA
agctgggacggcaaaggccttcactac

SEQ ID NO: 29 - DNA, 27 bp, unidentified
  source 1..27: mol_type = genomic DNA
gaggacagctagtacgactacatgttc

SEQ ID NO: 30 - DNA, 27 bp, unidentified
  source 1..27: mol_type = genomic DNA
aaccctttcttccctggctacgacctg

SEQ ID NO: 31 - DNA, 27 bp, unidentified
  source 1..27: mol_type = genomic DNA
agccactagacccagagctggcagtgg

SEQ ID NO: 32 - DNA, 27 bp, unidentified
  source 1..27: mol_type = genomic DNA
ggcaaaacctgggctggcaactgggag

SEQ ID NO: 33 - DNA, 27 bp, unidentified
  source 1..27: mol_type = genomic DNA
aacccttacttccctggcttcgacctg

SEQ ID NO: 34 - DNA, 30 bp, unidentified
  source 1..30: mol_type = genomic DNA
gtgtggatctgggagctgaacatgcacgac

SEQ ID NO: 35 - DNA, 21 bp, unidentified
  source 1..21: mol_type = genomic DNA
atgccttggtacgctgacctg

SEQ ID NO: 36 - DNA, 27 bp, unidentified
  source 1..27: mol_type = genomic DNA
agcctgcacagcgacgacaaagcttgg

SEQ ID NO: 37 - DNA, 21 bp, unidentified
  source 1..21: mol_type = genomic DNA
cctgcttggctggagggcctg

SEQ ID NO: 38 - DNA, 27 bp, unidentified
  source 1..27: mol_type = genomic DNA
aaccctttctaccctggcttcgacctg

SEQ ID NO: 39 - DNA, 30 bp, unidentified
  source 1..30: mol_type = genomic DNA
gtgtgggacaacaaacgctggctgcacgtg

SEQ ID NO: 40 - DNA, 27 bp, unidentified
  source 1..27: mol_type = genomic DNA
aacccttacttccctggctacgacctg

SEQ ID NO: 41 - DNA, 27 bp, unidentified
  source 1..27: mol_type = genomic DNA
aaatgggacggccgcggcatccacaaa

SEQ ID NO: 42 - DNA, 27 bp, unidentified
  source 1..27: mol_type = genomic DNA
gtgtgggtgaccggcagcgctcaccac

SEQ ID NO: 43 - DNA, 27 bp, unidentified
  source 1..27: mol_type = genomic DNA
agcatgtggaccgaccctatggctcac

SEQ ID NO: 44 - DNA, 27 bp, unidentified
  source 1..27: mol_type = genomic DNA
acctgggacggcaacggcgctcacttc

SEQ ID NO: 45 - DNA, 27 bp, unidentified
  source 1..27: mol_type = genomic DNA
cctccttggcttatggacttcgagggc

SEQ ID NO: 46 - DNA, 30 bp, unidentified
  source 1..30: mol_type = genomic DNA
atctgggacaaagaccgcaaaatgcaccac

SEQ ID NO: 47 - DNA, 27 bp, unidentified
  source 1..27: mol_type = genomic DNA
agcatctggaccgaccctatggctcac

SEQ ID NO: 48 - DNA, 27 bp, unidentified
  source 1..27: mol_type = genomic DNA
gtgtgggtgaccggcagccctcacgac

SEQ ID NO: 49 - DNA, 21 bp, unidentified
  source 1..21: mol_type = genomic DNA
cctccttggctggagggcttc

SEQ ID NO: 50 - DNA, 27 bp, unidentified
  source 1..27: mol_type = genomic DNA
aacccttactaccctggcttcgacctg

SEQ ID NO: 51 - DNA, 27 bp, unidentified
  source 1..27: mol_type = genomic DNA
agccagcacagcgacgacaaagcttgg

SEQ ID NO: 52 - DNA, 21 bp, unidentified
  source 1..21: mol_type = genomic DNA
atgccttggttcgctgacctg

SEQ ID NO: 53 - DNA, 27 bp, unidentified
  source 1..27: mol_type = genomic DNA
agctgggatggcaaaggcgctcacttc

SEQ ID NO: 54 - DNA, 27 bp, unidentified
  source 1..27: mol_type = genomic DNA
aaatgggacggccgcggcatgcacatc

SEQ ID NO: 55 - DNA, 27 bp, unidentified
  source 1..27: mol_type = genomic DNA
aaccctttctaccctggctacgacctg

SEQ ID NO: 56 - DNA, 27 bp, unidentified
  source 1..27: mol_type = genomic DNA
aactgggacggccgcggcatgcacaaa

SEQ ID NO: 57 - DNA, 27 bp, unidentified
  source 1..27: mol_type = genomic DNA
aaatgggacggccgcggcatccacaac

SEQ ID NO: 58 - DNA, 21 bp, unidentified
  source 1..21: mol_type = genomic DNA
ccttactggctggagggcctg

SEQ ID NO: 59 - DNA, 30 bp, unidentified
  source 1..30: mol_type = genomic DNA
gtgtggctggaggctcagaaactgcactac

SEQ ID NO: 60 - DNA, 27 bp, unidentified
  source 1..27: mol_type = genomic DNA
cctgagtggctggacgtgggcggctgg

SEQ ID NO: 61 - DNA, 27 bp, unidentified
  source 1..27: mol_type = genomic DNA
aacccttactaccctggctacgacctg

SEQ ID NO: 62 - DNA, 21 bp, unidentified
  source 1..21: mol_type = genomic DNA
cctccttggctgtacgacttc

SEQ ID NO: 63 - DNA, 27 bp, unidentified
  source 1..27: mol_type = genomic DNA
acctgggacggcaacggccctcacttc

SEQ ID NO: 64 - DNA, 27 bp, unidentified
  source 1..27: mol_type = genomic DNA
agctgggacggcaaaggtgctcacttc

SEQ ID NO: 65 - DNA, 27 bp, unidentified
  source 1..27: mol_type = genomic DNA
cctatgcacatggagctggagaaccag

SEQ ID NO: 66 - DNA, 21 bp, unidentified
  source 1..21: mol_type = genomic DNA
cctccttggctgcacgacctg

SEQ ID NO: 67 - DNA, 27 bp, unidentified
  source 1..27: mol_type = genomic DNA
agcatgtggagcgaccctatggctgac

SEQ ID NO: 68 - DNA, 28 bp, unidentified
  source 1..28: mol_type = genomic DNA
aaagtgagcgagctgcgctgtgcctagc

SEQ ID NO: 69 - DNA, 27 bp, unidentified
  source 1..27: mol_type = genomic DNA
ttcctgaacagcgagatgctgcacgag

SEQ ID NO: 70 - DNA, 27 bp, unidentified
  source 1..27: mol_type = genomic DNA
acctgggacggcaacggcgctcactac

SEQ ID NO: 71 - DNA, 27 bp, unidentified
  source 1..27: mol_type = genomic DNA
aactgggacggccgcggcatgcacaac

SEQ ID NO: 72 - DNA, 26 bp, unidentified
  source 1..26: mol_type = genomic DNA
gaccagtgggcagcatcgagaaagac

SEQ ID NO: 73 - DNA, 27 bp, unidentified
  source 1..27: mol_type = genomic DNA
agcatgtggagcgaccctagggctcac

SEQ ID NO: 74 - DNA, 27 bp, unidentified
  source 1..27: mol_type = genomic DNA
gagccttggtggatccagctggtggac

SEQ ID NO: 75 - DNA, 27 bp, unidentified
  source 1..27: mol_type = genomic DNA
atgttcaacatcgtgaaatgggcttac

SEQ ID NO: 76 - DNA, 27 bp, unidentified
  source 1..27: mol_type = genomic DNA
cctatgcacatggtgctggagaaccag

SEQ ID NO: 77 - protein, 9 aa, unidentified
SWDGKGAHF

SEQ ID NO: 78 - protein, 9 aa, unidentified
RKTWAGNWE

SEQ ID NO: 79 - protein, 9 aa, unidentified
TWDGKGAHF

SEQ ID NO: 80 - protein, 9 aa, unidentified
SMWSDPMAH

SEQ ID NO: 81 - protein, 7 aa, unidentified
PPWLADI

SEQ ID NO: 82 - protein, 9 aa, unidentified
TWDGKGAHY

SEQ ID NO: 83 - protein, 9 aa, unidentified
SWDGKGAHY

SEQ ID NO: 84 - protein, 9 aa, unidentified
VWVTGSAHD

SEQ ID NO: 85 - protein, 9 aa, unidentified
KWDGRGMHK

SEQ ID NO: 86 - protein, 9 aa, unidentified
SWDGKGPHF

SEQ ID NO: 87 - protein, 9 aa, unidentified
TWDGKGPHF

SEQ ID NO: 88 - protein, 9 aa, unidentified
KWDGRGMHN

SEQ ID NO: 89 - protein, 7 aa, unidentified
APWLEGL

SEQ ID NO: 90 - protein, 9 aa, unidentified
SIWSDPMAH

SEQ ID NO: 91 - protein, 7 aa, unidentified
APWLMDL

SEQ ID NO: 92 - protein, 7 aa, unidentified
PAWLEDL

SEQ ID NO: 93 - protein, 7 aa, unidentified
EDWHWPL

SEQ ID NO: 94 - protein, 10 aa, unidentified
IWDNDRKMHH

SEQ ID NO: 95 - protein, 9 aa, unidentified
SWDGKGPHY

SEQ ID NO: 96 - protein, 9 aa, unidentified
TWDGKGPHY

SEQ ID NO: 97 - protein, 7 aa, unidentified
PPWLVGL

SEQ ID NO: 98 - protein, 9 aa, unidentified
FLNSEMLHV

SEQ ID NO: 99 - protein, 9 aa, unidentified
RKTUAGNWE

SEQ ID NO: 100 - protein, 11 aa, unidentified
VRYGVLNAYTR

SEQ ID NO: 101 - protein, 11 aa, unidentified
MDFWPMSQQLA

SEQ ID NO: 102 - protein, 9 aa, unidentified
NPFFPGFDL

SEQ ID NO: 103 - protein, 7 aa, unidentified
APWLQDF

SEQ ID NO: 104 - protein, 9 aa, unidentified
SWDGKGLHY

SEQ ID NO: 105 - protein, 9 aa, unidentified
EDSUYDYMF

SEQ ID NO: 106 - protein, 9 aa, unidentified
NPFFPGYDL

SEQ ID NO: 107 - protein, 9 aa, unidentified
SHUTQSWQW

SEQ ID NO: 108 - protein, 9 aa, unidentified
GKTWAGNWE

SEQ ID NO: 109 - protein, 9 aa, unidentified
NPYFPGFDL

SEQ ID NO: 110 - protein, 10 aa, unidentified
VWIWELNMHD

SEQ ID NO: 111 - protein, 7 aa, unidentified
MPWYADL

SEQ ID NO: 112 - protein, 9 aa, unidentified
SLHSDDKAW

SEQ ID NO: 113 - protein, 7 aa, unidentified
PAWLEGL

SEQ ID NO: 114 - protein, 9 aa, unidentified
NPFYPGFDL

SEQ ID NO: 115 - protein, 10 aa, unidentified
VWDNKRWLHV

SEQ ID NO: 116 - protein, 9 aa, unidentified
NPYFPGYDL

SEQ ID NO: 117 - protein, 9 aa, unidentified
KWDGRGIHK

SEQ ID NO: 118 - protein, 9 aa, unidentified
VWVTGSAHH

SEQ ID NO: 119 - protein, 9 aa, unidentified
SMWTDPMAH

SEQ ID NO: 120 - protein, 9 aa, unidentified
TWDGNGAHF

SEQ ID NO: 121 - protein, 9 aa, unidentified
PPWLMDFEG

SEQ ID NO: 122 - protein, 10 aa, unidentified
IWDKDRKMHH

SEQ ID NO: 123 - protein, 9 aa, unidentified
SIWTDPMAH

SEQ ID NO: 124 - protein, 9 aa, unidentified
VWVTGSPHD

SEQ ID NO: 125 - protein, 7 aa, unidentified
PPWLEGF

SEQ ID NO: 126 - protein, 9 aa, unidentified
NPYYPGFDL

SEQ ID NO: 127 - protein, 9 aa, unidentified
SQHSDDKAW

SEQ ID NO: 128 - protein, 7 aa, unidentified
MPWFADL

SEQ ID NO: 129 - protein, 9 aa, unidentified
SWDGKGAHF

SEQ ID NO: 130 - protein, 9 aa, unidentified
KWDGRGMHI

SEQ ID NO: 131 - protein, 9 aa, unidentified
NPFYPGYDL

SEQ ID NO: 132 - protein, 9 aa, unidentified
NWDGRGMHK

SEQ ID NO: 133 - protein, 9 aa, unidentified
KWDGRGIHN

SEQ ID NO: 134 - protein, 7 aa, unidentified
PYWLEGL

SEQ ID NO: 135 - protein, 10 aa, unidentified
VWLEAQKLHY

SEQ ID NO: 136 - protein, 9 aa, unidentified
PEWLDVGGW

SEQ ID NO: 137 - protein, 9 aa, unidentified
NPYYPGYDL

SEQ ID NO: 138 - protein, 7 aa, unidentified
PPWLYDF

SEQ ID NO: 139 - protein, 9 aa, unidentified
TWDGNGPHF

SEQ ID NO: 140 - protein, 9 aa, unidentified
SWDGKGAHF

SEQ ID NO: 141 - protein, 9 aa, unidentified
PMHMELENQ

SEQ ID NO: 142 - protein, 7 aa, unidentified
PPWLHDL

SEQ ID NO: 143 - protein, 9 aa, unidentified
SMWSDPMAD

SEQ ID NO: 144 - protein, 9 aa, unidentified
KVSELRCAU

SEQ ID NO: 145 - protein, 9 aa, unidentified
FLNSEMLHE

SEQ ID NO: 146 - protein, 9 aa, unidentified
TWDGNGAHY

SEQ ID NO: 147 - protein, 9 aa, unidentified
NWDGRGMHN

SEQ ID NO: 148 - protein, 8 aa, unidentified
DQWAASRK

SEQ ID NO: 149 - protein, 9 aa, unidentified
SMWSDPRAH

SEQ ID NO: 150 - protein, 9 aa, unidentified
EPWWIQLVD

SEQ ID NO: 151 - protein, 9 aa, unidentified
MFNIVKWAY

SEQ ID NO: 152 - protein, 9 aa, unidentified
PMHMVLENQ

(19) United States
(12) Patent Application Publication
(10) Pub. No.: US 2024/0402182 A1
(43) Pub. Date: Dec. 5, 2024

(54) PEPTIDE TARGETING FUSOBACTERIA, COMPOSITION FOR DIAGNOSING CANCER COMPRISING SAME, AND DRUG DELIVERY COMPOSITION

(71) Applicant: INDUSTRY-ACADEMIC COOPERATION FOUNDATION, YONSEI UNIVERSITY, Seoul (KR)

(72) Inventors: Han Sang KIM, Seoul (KR); Kyung-A KIM, Seoul (KR); Joong Bae AHN, Seoul (KR); Soon Myung PAIK, Seoul (KR); Dong Eun YONG, Seoul (KR); Hyukmin LEE, Goyang-si, Gyeonggi-do (KR)

(73) Assignee: INDUSTRY-ACADEMIC COOPERATION FOUNDATION, YONSEI UNIVERSITY, Seoul (KR)

(21) Appl. No.: 18/699,219

(22) PCT Filed: Oct. 11, 2022
(86) PCT No.: PCT/KR2022/015317

§ 371 Date: Apr. 5, 2024

(30) Foreign Application Priority Data
Oct. 8, 2021 (KR) 10-2021-0133821

(51) Int. Cl. G01N 33/574; C12N 7/00; G01N 33/569

(52) U.S. Cl. CPC G01N 33/57488; C12N 7/00; G01N 33/56911; C12N 2795/14021; C12N 2795/14022; G01N 2333/01

(57) ABSTRACT

Provided are: a peptide targeting Fusobacteria, comprising a peptide consisting of an amino acid sequence selected from the group consisting of SEQ ID NOs: 77 to 83; a composition for diagnosing cancer, comprising same; and a drug delivery composition. The peptide targeting Fusobacteria accurately targets Fusobacteria, which are harmful bacteria in various diseases, including cancer, and thus can be effectively used in cancer diagnosis and drug delivery.

### TECHNICAL FIELD

The present invention relates to a peptide targeting Fusobacteria, a composition for diagnosing cancer comprising the same, and a drug delivery composition.

### BACKGROUND ART

Colon cancer or colorectal cancer is a malignant tumor that occurs in the appendix, colon, and rectum and a cancer that occurs in the mucosa, which is the innermost surface of the colon. The colon cancer is able to be confirmed only when cancer cells are found by a biopsy through colonoscopy. Most colon cancer has no symptoms in the early stages, so that diagnosis is quite difficult, and there is currently no method for predicting colon cancer in a non-invasive manner. With conventional diagnostic methods, since solid cancers such as colon cancer are often discovered in advanced stages, in order to prevent medical costs and deaths caused by colon cancer, an efficient method is to provide a method for preventing the occurrence of colon cancer in a high-risk group by predicting the occurrence and causative factors of colon tumors in advance.

Furthermore, the colon cancer includes a complex mixture of malignant cells, non-transformed cells, and microorganisms, and among them, the microorganisms, that is, pathogenic bacteria, can further worsen the stage of colon cancer. In addition, the presence or absence of colon cancer may also be determined through pathogenic bacteria.

The background art of the invention has been prepared to more facilitate understanding of the present invention. It should not be understood that the matters described in the background art of the invention exist as prior arts.

### DETAILED DESCRIPTION OF THE INVENTION

### Technical Problem

Recently, the use of bacteriophages has received great attention as a coping plan for bacterial diseases. In particular, interest in bacteriophages can be higher than ever due to the preference for nature-friendly methods. The bacteriophages are very small microorganisms that infect bacteria and are often abbreviated as phages.

The bacteriophages have the ability to proliferate inside bacterial cells after infection with bacteria, and kill bacteria by destroying the cell walls of the host bacteria when progeny bacteriophages come out of the bacteria after proliferation. A bacterial infection method of bacteriophages is very high specific, so that the types of bacteriophages that cancan infect specific bacteria are partially limited.

In other words, specific bacteriophages cancan infect only specific categories of bacteria, and as a result, the specific bacteriophages kill only specific bacteria and do not affect other bacteria.

Meanwhile, it has been known that the number of microorganisms living in the human body reaches 100 trillion, which is 10 times more than human cells, and the number of genes in the microorganisms is more than 100 times that of human genes. Microbiota or microbiome refers to the microbial community, including bacteria, archaea, and eukarya, present in given habitats, and it is known that gut microflora plays an important role in human physiology and has a significant impact on human health and disease through interactions with human cells.

Among these symbiotic microorganisms, Fusobacterium nucleatum is one of the most prevalent bacterial species in these colon cancer tissues and can contribute to the development of colon cancer. More specifically, Fusobacteria are associated with human colorectal cancer, and 40 to 60% of tumor tissues from colorectal cancer patients are infected with Fusobacteria. The Fusobacteria are selectively present in tumor cells at a high concentration of 10 times higher than in surrounding cells, which can expand the number of tumor-promoting bone marrow cells, activate a β-catenin-WNT signaling mechanism as a binding reaction of FadA adhesin to E-cadherin of host bacteria, and alter anticancer suppressive immune responses by promoting immunoglobulins on T cells and NK cells through a bacterial Fap2 protein and T cell immunoreceptors with an immunoreceptor tyrosine-based inhibitory motif domain.

Accordingly, the present inventors focused on Fusobacteria in the diagnosis and treatment of colon cancer, and recognized that when the above-described bacteriophages are applied to Fusobacteria, it is possible to more efficiently treat and diagnose various carcinomas including colon cancer. In particular, Fusobacteria as anaerobic bacteria are selectively present in cancer cell tissue having a lower oxygen concentration than the surrounding tissue, and have the selectivity of more than 10 times to label tumor tissue, and are expected to have an advantage of being developed as a treatment.

Ultimately, the present inventors found specific peptide sequences capable of selectively targeting Fusobacteria and then developed a composition for diagnosing various carcinomas including colon cancer, by administering the synthesized peptide sequences to a subject or a drug delivery composition capable of being used to treat carcinomas.

Therefore, an object of the present invention is to provide a composition for diagnosing various carcinomas including colon cancer, or a drug delivery composition for treating various carcinomas, using peptides having selectivity to Fusobacteria.

Furthermore, another object of the present invention is to provide a composition for treating various carcinomas including colon cancer by displaying peptides having selectivity to Fusobacteria on bacteriophages to selectively kill Fusobacteria.

The objects of the present invention are not limited to the aforementioned objects, and other objects, which are not mentioned above, will be apparent to a person having ordinary skill in the art from the following description.

### Technical Solution

An aspect of the present invention provides a peptide targeting Fusobacteria including a peptide consisting of an amino acid sequence selected from the group consisting of SEQ ID NO: 77 to SEQ ID NO: 83, in which the peptide targets Fusobacteria.

A nucleic acid sequence of SEQ ID NOs: 1 to 76 and an amino acid sequence of a single peptide consisting of SEQ ID NOs: 77 to 152 of the present invention found sequences selective to Fusobacteria using a M13 bacteriophage peptide library (a peptide library designed to have 9 amino acid sequences, and having approximately 8.60×10¹⁰ amino acid sequences).

According to the feature of the present invention, the above-described peptide can be displayed in a major coat protein P3 of the M13 bacteriophage.

Another aspect of the present invention provides an expression vector including one of sequences represented by SEQ ID NOs: 1 to 7 encoding an amino acid sequence selected from the group consisting of SEQ ID NOs: 77 to 83.

According to the feature of the present invention, the above-described expression vector can be a phagemid vector.

Yet another aspect of the present invention provides a transformant transformed with the above-described expression vector.

Yet another aspect of the present invention provides a composition for diagnosing cancer, including a peptide targeting Fusobacteria consisting of an amino acid sequence selected from the group consisting of SEQ ID NOs: 77 to 83.

According to the feature of the present invention, the above-described peptide targeting Fusobacteria can be labeled with one selected from the group consisting of a color enzyme, a radioisotope, a chromophore, a luminescent substance, a fluorescer, super paramagnetic particles, and ultrasuper paramagnetic particles.

According to the feature of the present invention, the above-described peptide targeting Fusobacteria can target and diagnose at least one cancer cell of colorectal cancer, melanoma, pancreatic cancer, liver cancer, stomach cancer, colon cancer, lung cancer, ovarian cancer, breast cancer, and cervical cancer infected with Fusobacteria.

Yet another aspect of the present invention provides a drug delivery composition, including a peptide targeting Fusobacteria consisting of an amino acid sequence selected from the group consisting of SEQ ID NOs: 77 to 83.

According to the feature of the present invention, the above-described drug can be physically or chemically bound to the peptide targeting Fusobacteria, and can be an anticancer agent for treating cancer cells infected with Fusobacteria.

According to the feature of the present invention, the above-described anticancer agent can target and treat at least one cancer cell of colorectal cancer, melanoma, pancreatic cancer, liver cancer, stomach cancer, colon cancer, lung cancer, ovarian cancer, breast cancer, and cervical cancer infected with Fusobacteria.

According to the feature of the present invention, the above-described anticancer agent can be selected from the group consisting of Leucovorin, Levamisole, Irinotecan, Oxaliplatin, Capecitabine, Uracil/Tegafur, Docetaxel, cis-platin, camptothecin, paclitaxel, Tamoxifen, Anasterozole, Gleevec, 5-fluorouracil (5-FU), Floxuridine, Leuprolide, Flutamide, Zoledronate, Doxorubicin, Vincristine, Gemcitabine, Streptozocin, Carboplatin, Topotecan, Belotecan, Vinorelbine, hydroxyurea, nitrosourea, Valrubicin, retinoic acid series, Methotrexate, Mechlorethamine, Chlorambucil, Busulfan, Doxifluridine, Vinblastin, Mitomycin, Prednisone, Testosterone, Mitoxantron, aspirin, salicylates, ibuprofen, naproxen, fenoprofen, indomethacin, phenylbutazone, cyclophosphamide, mechlorethamine, dexamethasone, prednisolone, celecoxib, valdecoxib, nimesulide, daunorubicin, actinomycin-D, etoposide, teniposide, bisantrene, homoharringtonine, busulfan, chlorambucil, melphalan, nitrogen mustard, cortisone, and corticosteroid.

According to the feature of the present invention, the above-described anticancer agent can be physically or chemically bound to the peptide targeting Fusobacteria, and can be a gene drug for treating cancer cells infected with Fusobacteria.

According to the feature of the present invention, the above-described gene drug can be selected from the group consisting of small interfering RNA (siRNA) and single guide RNA (sgRNA) targeting at least one protein selected from the group consisting of Bcl-2 Antagonist X (Bax), B-cell lymphoma 2 (BCl-2), Focal adhesion kinase, Matrix metalloproteinase, Vascular endothelial growth factor (VEGF), Fatty acid synthase, Multiple drug resistance protein (MDR), Harvey rat sarcoma viral oncogene homolog (H-Ras), Kirsten-rat sarcoma viral oncogene homolog (K-Ras), Polo Like Kinase 1 (PLK-1), Transforming growth factor beta (TGF-β), Signal Transducer And Activator Of Transcription 3 (STAT3), Epidermal growth factor receptor (EGFR), Protein kinase C alpha (PKC-α), Epstein-Barr virus, human papillomavirus E6 (HPV E6), BCR-ABL fusion gene and Telomerase of cancer cells.

Hereinafter, the present invention will be described in more detail with reference to Examples. However, these Examples are only illustrative the present invention, and the scope of the present invention is not limited to these Examples.

### Effects of the Invention

According to the present invention, it is possible to target Fusobacteria with high affinity, thereby accurately targeting Fusobacteria, which is a harmful bacterium in various diseases including cancer.

Moreover, it is possible to target Fusobacteria with high affinity as described above, thereby detecting the presence or absence of Fusobacteria with high reliability.

Accordingly, when the peptide having selectivity to Fusobacteria according to an embodiment of the present invention and a bacteriophage or peptide expressing the peptide having selectivity to Fusobacteria are used as a kit for detecting Fusobacteria, for example, the distribution and presence or absence of metastasis of cancer cells infected with Fusobacteria can be confirmed in the form of an RT-PCR kit, a DNA chip kit, or a protein chip kit.

When the peptide having selectivity to Fusobacteria according to an embodiment of the present invention is a drug delivery system, and delivers a chemical anti-cancer therapeutic agent or genetic drug into cancer cells infected with Fusobacteria, the aforementioned chemical anti-cancer therapeutic agent and genetic drug can invade the inside of cancer cells, improve more efficiently drug delivery, and as a result, also improve the effect of anti-cancer treatment.

The effects of the present invention are not limited by the foregoing, and other various effects are anticipated in the present invention.

### BRIEF DESCRIPTION OF DRAWINGS

FIG. 1 illustrates a distribution of bacteria in various carcinomas.

FIG. 2A is a schematic diagram listing types of Fusobacteria used to screen bacteriophage peptides having selectivity to Fusobacteria in the present invention.

FIG. 2B is a schematic diagram of a phage display (M13 Bacteriophage display) method used to screen bacteriophage peptides having selectivity to Fusobacteria.

FIG. 3 is a schematic diagram of amino acid sequences and sequences of bacteriophage peptides having selectivity to Fusobacteria according to an embodiment of the present invention.

FIGS. 4A to 4E illustrate sequences and amino acid sequences having selectivity to Fusobacteria according to an embodiment of the present invention (FIG. 4A); an expression pCD-TriC9 phagemid vector map used to display a protein selective to Fusobacteria in a coat protein of M13 bacteriophage (FIG. 4B); gene cloning PCR for sequences selective to Fusobacteria (FIG. 4C); an enzyme-linked immunosorbent assay (ELISA) result of testing the binding affinity of bacteriophages containing peptide sequences selective to Fusobacteria (FIG. 4D); and ELISA results of testing the binding affinity according to a type of Fusobacteria (FIG. 4E).

FIGS. 5A to 5I are diagrams illustrating the detection efficacy of bacteria using peptides of SEQ ID NOs: 77 to 83 bound with fluorescent labeling factors.

FIGS. 6 to 8 are schematic diagrams for structures of bacteriophages used in the present invention.

### BEST MODES FOR CARRYING OUT THE INVENTION

Advantages and features of the present invention, and methods for accomplishing the same will be more clearly understood from exemplary embodiments described in detail below with reference to the accompanying drawings. However, the present invention is not limited to the exemplary embodiments set forth below, and will be embodied in various different forms. The exemplary embodiments are just for rendering the disclosure of the present invention complete and are set forth to provide a complete understanding of the scope of the invention to those skilled in the art to which the present invention pertains.

In this specification, expressions such as “have”, “can have”, “include”, or “can include” refer to the presence of the corresponding feature (e.g., numerical value, function, operation, or component such as part), and does not exclude the presence of additional features.

In the present invention, the expression such as “A or B”, “at least one of A and/or B”, or “one or more of A and/or B” can include all possible combinations of items listed together. For example, “A or B”, “at least one of A and B”, or “at least one of A or B” can refer to all cases of (1) including at least one A, (2) including at least one B, or (3) including both at least one A and at least one B.

The expression of “configured to” used herein can be changed and used to, for example, “suitable for”, “having the capacity to”, “designed to”, “adapted to”, “made to” or “capable of”, depending on the situation.

The terms used herein are used to illustrate only specific exemplary embodiments, and can not be intended to limit the scope of other exemplary embodiments. A singular form can include a plural form unless otherwise clearly meant in the contexts. The terms used herein, including technical or scientific terms, can have the same meaning as generally understood by those of ordinary skill in the art described in the present invention. The terms defined in a general dictionary among the terms used herein can be interpreted in the same or similar meaning as or to the meaning on the context of the related art, and will not be interpreted as an ideal or excessively formal meaning unless otherwise defined in the present invention. In some cases, even the terms defined in the present invention can not be interpreted to exclude the exemplary embodiments of the present invention.

The features of various embodiments of the present invention can be partially or entirely bonded to or combined with each other and may be interlocked and operated in technically various ways, and the embodiments may be carried out independently of or in association with each other.

As used herein, the term “individual” can mean animals, including humans, with cancer diseases including colon cancer, and can be used interchangeably with terms such as a host, a subject, and a patient.

Furthermore, the cancer disease can include at least one selected from the group consisting of colorectal cancer, melanoma, pancreatic cancer, liver cancer, stomach cancer, colon cancer, lung cancer, ovarian cancer, breast cancer, and cervical cancer, infected with Fusobacteria.

As used in the present invention, the term “peptide” refers to an amino acid chain linked by multiple peptide bonds, and can be used interchangeably with terms such as a protein, a polypeptide, or an amino acid.

As used in the present invention, the term “expression vector” refers to a nucleic acid used for inserting or using foreign DNA, and can be used interchangeably with a cloning vector, a plasmid, or a phagemid.

In the present invention, transformation with the expression vector can be performed by transformation techniques known to those skilled in the art. Preferably, the transformation technique can be used with microprojectile bombardment, electroporation, calcium phosphate (CaPO₄) precipitation, calcium chloride (CaCl₂)) precipitation, PEG-mediated fusion, microinjection, and a liposome-mediated method, and the transformant can be Escherichia coli, Bacillus subtilis, Streptomyces, Pseudomonas, Proteus mirabilis, Staphylococcus, and Agrobacterium tumefaciens, but is not limited thereto.

As used in the present invention, the term “diagnosis of colon cancer” means determining whether a patient is likely to develop colon cancer, whether the likelihood of developing colon cancer is relatively high, or whether colon cancer has already developed. The method of the present invention can be used to delay the onset of colon cancer or prevent the development through special and appropriate management for any specific patient at high risk of developing colon cancer. In addition, the method of the present invention can be used clinically to diagnose colon cancer at an early stage and determine treatment by selecting the most appropriate treatment method.

Hereinafter, the present invention will be described in more detail through Examples. However, these Examples are intended to illustrate one or more exemplary embodiments, and the scope of the present invention is not limited to these Examples.

### Example 1: Selection of Bacteriophage Peptides Having Selectivity to Fusobacteria According to an Embodiment of the Present Invention

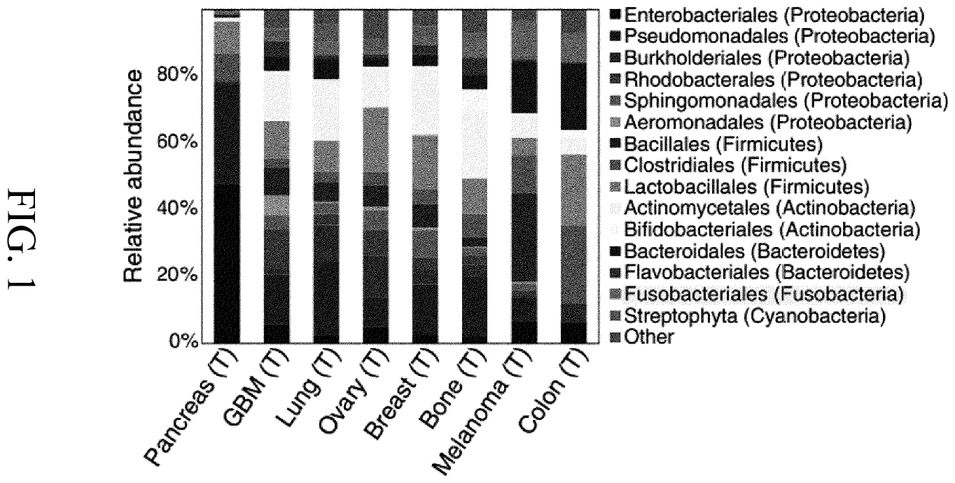

FIG. 1 illustrates a distribution of bacteria in various carcinomas.

Referring to FIG. 1, there is illustrated a distribution map according to carcinomas of Fusobacteria targeted by the bacteriophage according to an embodiment of the present invention, and Fusobacteria are found in various carcinomas including pancreas cancer, glioblastoma (GBM), lung cancer, ovary cancer, breast cancer, bone cancer, melanoma, colon cancer, and the like.

The Fusobacteria are anaerobic bacteria found only in the oral cavity. However, in the case of cancer cells, as oxygen consumption is higher than that of normal cells and a hypoxic environment is induced, Fusobacteria can move to the area of the cancer cells where the hypoxic environment is created to form colonies thereof. Accordingly, when targeting Fusobacteria to areas other than the oral cavity, the distribution and presence or absence of cancer cells, and even metastasis of cancer cells, can be confirmed.

Therefore, the present inventors selected Fusobacteria as target bacteria for the treatment and diagnosis of various carcinomas, including colon cancer.

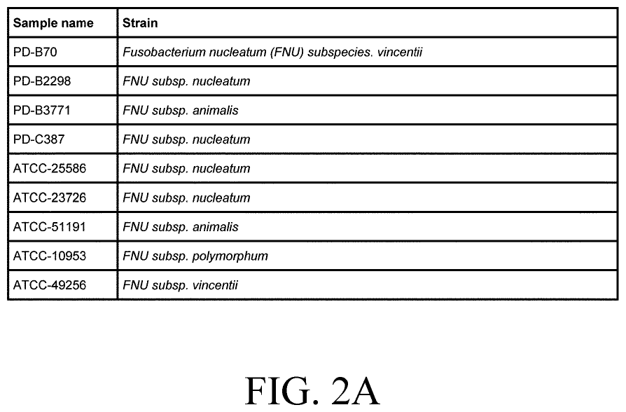

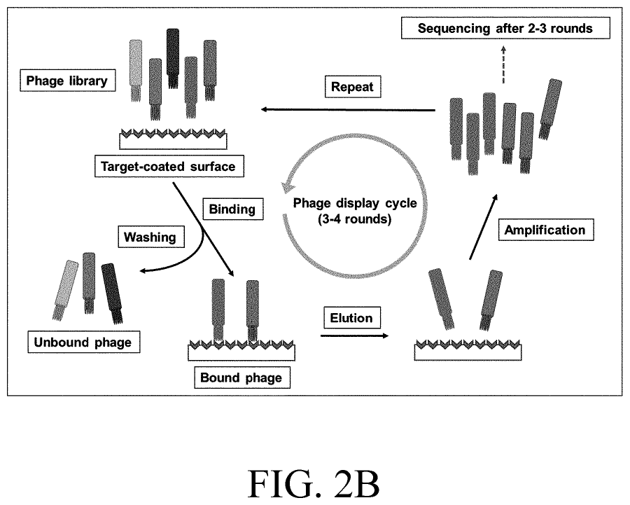

FIG. 2A is a schematic diagram of types of Fusobacteria.

Referring to FIG. 2A, in the present invention, a total of 9 types of Fusobacteria were used to select bacteriophage peptides having Fusobacteria selectivity. Among them, Fusobacterium nucleatum subspecies vincentii (PD-B70), Fusobacterium nucleatum subspecies nucleatum (PD-B2298), Fusobacterium nucleatum subspecies animalis (PD-B3771), and Fusobacterium nucleatum subspecies nucleatum (PD-C387) are Fusobacteria derived from patients, and Fusobacterium nucleatum subspecies nucleatum (ATCC-25586), Fusobacterium nucleatum subspecies nucleatum (ATCC-23726), Fusobacterium nucleatum subspecies animalis (ATCC-51191), Fusobacterium nucleatum subspecies polymorphum (ATCC-10953) and Fusobacterium nucleatum subspecies vincentii (ATCC-49256) are Fusobacteria subspecies commercially available from the American Type Culture Collection (ATCC).

Next, referring to FIG. 2B, a schematic diagram of a phage display method is illustrated, and the present invention screened bacteriophages capable of specifically detecting 9 types of Fusobacteria described above through phage display. The phage display method can discover molecular diagnostic peptides consisting of new sequences through biopanning.

First, in the present invention, 9 types of Fusobacteria described above are applied and brought into contact with various types of phage libraries, and then, through repeated washing steps, a total of 7 types of bacteriophage candidates based on M13 bacteriophage specifically bound to Fusobacteria were selected.

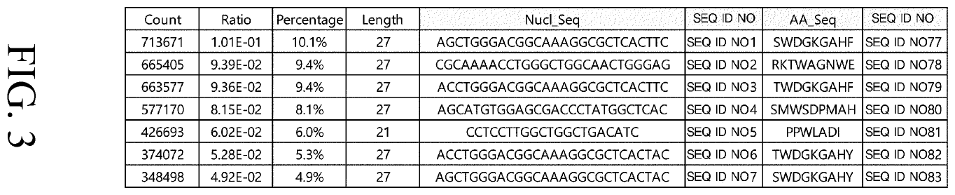

Accordingly, FIG. 3 illustrates a schematic diagram of sequences and amino acid sequences having selectivity to Fusobacteria according to an embodiment of the present invention.

At this time, the bacteriophage peptides having selectivity to Fusobacteria according to an embodiment of the present invention were identified through sequencing of the total of 7 bacteriophage candidates selected in FIG. 2B, and the sequencing was performed at Creative biolabs INC (USA).

More specifically, sequences at a DNA level for the bacteriophage peptides capable of specifically recognizing Fusobacteria used in the present invention can be the sequences represented by SEQ ID NO: 1 to 7, and the amino acid sequences at a protein level can be sequences represented by SEQ ID NOs: 77 to 83, but are not limited thereto, which are upper 14 sequences most frequently discovered, and the sequence at a DNA level and the amino acid sequences at a protein level can further include lower 138 sequences.

For example, the sequences of bacteriophages used in the present invention can include all of sequences shown in [Table 1] below.

TABLE 1

| | | | | | SEQ ID | | SEQ ID |
|---|---|---|---|---|---|---|---|
| Count | Ratio | Percentage | Length | Nucl_Seq | NO: | AA Seq | NO: |
| 713671 | 1.01E−01 | 10.1% | 27 | AGCTGGGACGGCAAAG | SEQ ID | SWDGK | SEQ ID |
| | | | | GCGCTCACTTC | NO: 1 | GAHF | NO: 77 |
| 665405 | 9.39E−02 | 9.4% | 27 | CGCAAAACCTGGGCTG | SEQ ID | RKTWA | SEQ ID |
| | | | | GCAACTGGGAG | NO: 2 | GNWE | NO: 78 |
| 663577 | 9.36E−02 | 9.4% | 27 | ACCTGGGACGGCAAAG | SEQ ID | TWDGK | SEQ ID |
| | | | | GCGCTCACTTC | NO: 3 | GAHF | NO: 79 |
| 577170 | 8.15E−02 | 8.1% | 27 | AGCATGTGGAGCGACC | SEQ ID | SMWSDP | SEQ ID |
| | | | | CTATGGCTCAC | NO: 4 | MAH | NO: 80 |
| 426693 | 6.02E−02 | 6.0% | 21 | CCTCCTTGGCTGGCTGA | SEQ ID | PPWLAD | SEQ ID |
| | | | | CATC | NO: 5 | I | NO: 81 |
| 374072 | 5.28E−02 | 5.3% | 27 | ACCTGGGACGGCAAAG | SEQ ID | TWDGK | SEQ ID |
| | | | | GCGCTCACTAC | NO: 6 | GARY | NO: 82 |
| 348498 | 4.92E−02 | 4.9% | 27 | AGCTGGGACGGCAAAG | SEQ ID | SWDGK | SEQ ID |
| | | | | GCGCTCACTAC | NO: 7 | GAHY | NO: 83 |
| 192353 | 2.71E−02 | 2.7% | 27 | GTGTGGGTGACCGGCA | SEQ ID | VWVTGS | SEQ ID |
| | | | | GCGCTCACGAC | NO: 8 | AHD | NO: 84 |
| 158578 | 2.24E−02 | 2.2% | 27 | AAATGGGACGGCCGCG | SEQ ID | KWDGR | SEQ ID |
| | | | | GCATGCACAAA | NO: 9 | GMHK | NO: 85 |
| 144588 | 2.04E−02 | 2.0% | 27 | AGCTGGGACGGCAAAG | SEQ ID | SWDGK | SEQ ID |
| | | | | GCCCTCACTTC | NO: 10 | GPHF | NO: 86 |
| 125123 | 1.77E−02 | 1.8% | 27 | ACCTGGGACGGCAAAG | SEQ ID | TWDGK | SEQ ID |
| | | | | GCCCTCACTTC | NO: 11 | GPHF | NO: 87 |
| 108760 | 1.53E−02 | 1.5% | 27 | AAATGGGACGGCCGCG | SEQ ID | KWDGR | SEQ ID |
| | | | | GCATGCACAAC | NO: 12 | GMHN | NO: 88 |
| 87916 | 1.24E−02 | 1.2% | 21 | GCTCCTTGGCTGGAGG | SEQ ID | APWLEG | SEQ ID |
| | | | | GCCTG | NO: 13 | L | NO: 89 |
| 78682 | 1.11E−02 | 1.1% | 27 | AGCATCTGGAGCGACC | SEQ ID | SIWSDP | SEQ ID |
| | | | | CTATGGCTCAC | NO: 14 | MAH | NO: 90 |
| 55246 | 7.80E−02 | 0.8% | 21 | GCTCCTTGGCTGATGGA | SEQ ID | APWLM | SEQ ID |
| | | | | CCTG | NO: 15 | DL | NO: 91 |
| 52922 | 7.47E−02 | 0.7% | 21 | CCTGCTTGGCTGGAGG | SEQ ID | PAWLED | SEQ ID |
| | | | | ACCTG | NO: 16 | L | NO: 92 |
| 52856 | 7.46E−03 | 0.7% | 21 | GAGGACTCGCACTGCC | SEQ ID | EDWHW | SEQ ID |
| | | | | CTCTG | NO: 17 | PL | NO: 93 |
| 50585 | 7.14E−03 | 0.7% | 30 | ATCTGCGACAACGACC | SEQ ID | IWDNDR | SEQ ID |
| | | | | GCAAAATGCACCAC | NO: 18 | KMHH | NO: 94 |
| 47516 | 6.71E−03 | 0.7% | 27 | AGCTGGGACGGCAAAG | SEQ ID | SWDGK | SEQ ID |
| | | | | CCCCTCACTAC | NO: 19 | GPHY | NO: 95 |
| 47136 | 6.65E−03 | 0.7% | 27 | ACCTCGGACGGCAAAC | SEQ ID | TWDGK | SEQ ID |
| | | | | GCCCTCACTAC | NO: 20 | GPHY | NO: 96 |
| 32606 | 4.60E−03 | 0.5% | 21 | CCTCCTTGGCTGCTGGG | SEQ ID | PPWLVG | SEQ ID |
| | | | | CCTG | NO: 21 | L | NO: 97 |
| 32519 | 4.59E−03 | 0.5% | 27 | TTCCTGAACAGCGAGA | SEQ ID | FLNSEM | SEQ ID |
| | | | | TCCTGCACGTG | NO: 22 | LHV | NO: 98 |
| 25339 | 3.58E−03 | 0.4% | 27 | CGCAAAACCTGAGCTC | SEQ ID | RKT*AG | SEQ ID |
| | | | | CCAACTGGGAG | NO: 23 | NWE | NO: 99 |
| 24889 | 3.51E−03 | 0.4% | 33 | GTGCGCTACGCCGTGCT | SEQ ID | VRYGVL | SEQ ID |
| | | | | GAACGCTTACACCCGC | NO: 24 | NAYTR | NO: 100 |
| 23121 | 3.26E−03 | 0.3% | 33 | ATGGACTTCTGGCCTAT | SEQ ID | MDFWP | SEQ ID |
| | | | | GAGCCAGCACCTGCCT | NO: 25 | MSQQLA | NO: 101 |
| 22836 | 3.22E−03 | 0.3% | 27 | AACCCTTTCTTCCCTGC | SEQ ID | NPFFPGF | SEQ ID |
| | | | | CTTCCACCTG | NO: 26 | DL | NO: 102 |
| 22130 | 3.12E−03 | 0.3% | 21 | CCTCCTTCGCTCCACCA | SEQ ID | APWLQD | SEQ ID |
| | | | | CTTC | NO: 27 | F | NO: 103 |
| 21502 | 3.03E−03 | 0.3% | 27 | AGCTGGGACGGCAAAG | SEQ ID | SWDGK | SEQ ID |
| | | | | GCCTTCACTAC | NO: 28 | GLHY | NO: 104 |
| 17502 | 2.47E−03 | 0.2% | 27 | GAGGACAGCTAGTACG | SEQ ID | EDS*YD | SEQ ID |
| | | | | ACTACATCTTC | NO: 29 | YMF | NO: 105 |
| 17190 | 2.43E−03 | 0.2% | 27 | AACCCTTTCTTCCCTGG | SEQ ID | NPFFPG | SEQ ID |
| | | | | CTACGACCTC | NO: 30 | YDL | NO: 106 |
| 16911 | 2.39E−03 | 0.2% | 27 | AGCCACTAGACCCAGA | SEQ ID | SH*TQS | SEQ ID |
| | | | | GCTGGCAGTGC | NO: 31 | WQW | NO: 107 |
| 16445 | 2.32E−03 | 0.2% | 27 | GGCAAAACCTCGCCTC | SEQ ID | GKTWA | SEQ ID |
| | | | | GCAACTGCGAG | NO: 32 | GNWE | NO: 108 |
| 16210 | 2.29E−03 | 0.2% | 27 | AACCCTTACTTCCCTGG | SEQ ID | NPYFPG | SEQ ID |
| | | | | CTTCGACCTG | NO: 33 | FDL | NO: 109 |
| 14563 | 2.06E−03 | 0.2% | 30 | GTGTGGATCTGGGAGC | SEQ ID | VWIWEL | SEQ ID |
| | | | | TGAACATCCACGAC | NO: 34 | NMHD | NO: 110 |
| 14450 | 2.04E−03 | 0.2% | 21 | ATGCCTTGGTACGCTGA | SEQ ID | MPWYA | SEQ ID |
| | | | | CCTC | NO: 35 | DL | NO: 111 |
| 14342 | 2.02E−03 | 0.2% | 27 | AGCCTGCACAGCGACG | SEQ ID | SLHSDD | SEQ ID |
| | | | | ACAAAGCTTGG | NO: 36 | KAW | NO: 112 |
| 11102 | 1.57E−03 | 0.2% | 21 | CCTGCTTGCCTGGAGG | SEQ ID | PAWLEG | SEQ ID |
| | | | | GCCTC | NO: 37 | | NO: 113 |
| 10514 | 1.48E−03 | 0.1% | 27 | AACCCTTTCTACCCTGG | SEQ ID | NPFYPG | SEQ ID |
| | | | | CTTCGACCTC | NO: 38 | FDL | NO: 114 |
| 10504 | 1.48E−03 | 0.1% | 30 | GTGTGGGACAACAAAC | SEQ ID | VWDNK | SEQ ID |
| | | | | GCTGGCTGCACCTC | NO: 39 | RWLAV | NO: 115 |
| 10465 | 1.48E−03 | 0.1% | 27 | AACCCTTACTTCCCTCG | SEQ ID | NPYFPG | SEQ ID |
| | | | | CCACGACCTG | NO: 40 | YDL | NO: 116 |
| 10402 | 1.47E−03 | 0.1% | 27 | AAATGCGACGGCCGCG | SEQ ID | KWDGR | SEQ ID |
| | | | | GCATCCACAAA | NO: 41 | GTNK | NO: 117 |
| 10221 | 1.44E−03 | 0.1% | 27 | GTGTGGGTGACCCCCA | SEQ ID | WVTGS | SEQ ID |
| | | | | GCGCTCACCAC | NO: 42 | AHH | NO: 118 |
| 9730 | 1.37E−03 | 0.1% | 27 | AGCAIGTGGACCGACC | SEQ ID | SMWTDP | SEQ ID |
| | | | | CTATCGCTCAC | NO: 43 | MAH | NO: 119 |
| 9488 | 1.34E−03 | 0.1% | 27 | ACCTGGGACGGCAACG | SEQ ID | TWDGN | SEQ ID |
| | | | | GCGCTCACTTC | NO: 44 | GAHF | NO: 120 |
| 9213 | 1.30E−03 | 0.1% | 27 | CCTCCTTGGCTTATGGA | SEQ ID | PPWLMD | SEQ ID |
| | | | | CTTCCAGGGC | NO: 45 | FEG | NO: 121 |
| 8599 | 1.21E−03 | 0.1% | 30 | ATCTCGGACAAAGACC | SEQ ID | IWDKDR | SEQ ID |
| | | | | CCAAAATGCACCAC | NO: 46 | KMHH | NO: 122 |
| 8199 | 1.16E−03 | 0.1% | 27 | AGCATCTGGACCGACC | SEQ ID | SIWTDP | SEQ ID |
| | | | | CTATCCCTCAC | NO: 47 | MAN | NO: 123 |
| 7777 | 1.10E−03 | 0.1% | 27 | GTGTGGGTGACCGGCA | SEQ ID | WVTGSI | SEQ ID |
| | | | | GCCCTCACGAC | NO: 48 | PHD | NO: 124 |
| 7651 | 1.08E−03 | 0.1% | 21 | CCTCCTTGGCTGGAGGG | SEQ ID | PPWLEO | SEQ ID |
| | | | | CTTC | NO: 49 | F | NO: 125 |
| 7281 | 1.03E−03 | 0.1% | 27 | AACCCTTACTACCCTGG | SEQ ID | NPYYPG | SEQ ID |
| | | | | CTTCGACCTG | NO: 50 | FDL | NO: 126 |
| 6531 | 9.22E−04 | 0.1% | 27 | AGCCAGCACAGCGACC | SEQ ID | SQHSDD | SEQ ID |
| | | | | ACAAAGCTTGG | NO: 51 | KAW | NO: 127 |
| 6333 | 8.94E−04 | 0.1% | 21 | ATGCCTTGCTTCGCTGA | SEQ ID | MPWFA | SEQ ID |
| | | | | CCTG | NO: 52 | DL | NO: 128 |
| 6305 | 8.90E−04 | 0.1% | 27 | AGCTGCGATGCCAAAG | SEQ ID | SWDGK | SEQ ID |
| | | | | CCGCTCACTTC | NO: 53 | GAHF | NO: 129 |
| 6295 | 8.88E−04 | 0.1% | 27 | AAATGGGACGCCCCCG | SEQ ID | KWDGR | SEQ ID |
| | | | | GCATGCACATC | NO: 54 | GMHI | NO: 130 |
| 6235 | 8.80E−04 | 0.1% | 27 | AACCCTTTCTACCCTGC | SEQ ID | NPFYPG | SEQ ID |
| | | | | CTACGACCTG | NO: 55 | YDL | NO: 131 |
| 6205 | 8.76E−04 | 0.1% | 27 | AACTGGGACGGCCGCG | SEQ ID | NWDGR | SEQ ID |
| | | | | GCATGCACAAA | NO: 56 | GMHK | NO: 132 |
| 6187 | 8.73E−04 | 0.1% | 27 | AAATGGGACGGCCGCC | SEQ ID | KWDGR | SEQ ID |
| | | | | GCATCCACAAC | NO: 57 | GIHN | NO: 133 |
| 6171 | 8.71E−04 | 0.1% | 21 | CCTTACTCGCTGGAGG | SEQ ID | PYWLEG | SEQ ID |
| | | | | GCCTG | NO: 58 | L | NO: 134 |
| 5917 | 8.35E−04 | 0.1% | 30 | GTGTCGCTCGAGGCTC | SEQ ID | VWLEA | SEQ ID |
| | | | | AGAAACTGCACTAC | NO: 59 | QKLHY | NO: 135 |
| 5908 | 8.34E−04 | 0.1% | 27 | CCTGAGTGGCTGGACG | SEQ ID | PEWLDV | SEQ ID |
| | | | | TCGGCGGCTGC | NO: 60 | GGW | NO: 136 |
| 5699 | 8.04E−04 | 0.1% | 27 | AACCCTTACTACCCTGG | SEQ ID | NPYYPG | SEQ ID |
| | | | | CTACGACCTG | NO: 61 | YDL | NO: 137 |
| 5680 | 8.02E−04 | 0.1% | 21 | CCTCCTTGCCTGTACGA | SEQ ID | PPWLYD | SEQ ID |
| | | | | CTTC | NO: 62 | F | NO: 138 |
| 5515 | 7.75E−04 | 0.1% | 27 | ACCTGGGACGCCAAGC | SEQ ID | TWDGN | SEQ ID |
| | | | | CCCCTCACTTC | NO: 63 | GPHF | NO: 139 |
| 5286 | 7.46E−04 | 0.1% | 27 | AGCTGGGACGGCAAAG | SEQ ID | SWDGK | SEQ ID |
| | | | | GTGCTCACTTC | NO: 64 | GAHF | NO: 140 |
| 4331 | 6.11E−04 | 0.1% | 27 | CCTATGCACATCGACCT | SEQ ID | PMHMEL | SEQ ID |
| | | | | GGAGAACCAG | NO: 65 | ENQ | NO: 141 |
| 4246 | 5.99E−04 | 0.1% | 21 | CCTCCTTGGCTGCACGA | SEQ ID | PPWLHD | SEQ ID |
| | | | | CCTG | NO: 66 | L | NO: 142 |
| 4141 | 5.84E−04 | 0.1% | 27 | AGCATGTGGAGCGACC | SEQ ID | SMWSDP | SEQ ID |
| | | | | CTATGGCTGAC | NO: 67 | MAD | NO: 143 |
| 4067 | 5.74E−04 | 0.1% | 28 | AAAGTGAGCGAGCTGC | SEQ ID | KVSELR | SEQ ID |
| | | | | GCTGTGCCTAGC | NO: 68 | CA* | NO: 144 |
| 4044 | 5.71E−04 | 0.1% | 27 | TTCCTGAACAGCGAGA | SEQ ID | FLNSEM | SEQ ID |
| | | | | TGCTGCACGAG | NO: 69 | LHE | NO: 145 |
| 4043 | 5.71E−04 | 0.1% | 27 | ACCTGGGACGGCAACG | SEQ ID | TWDGN | SEQ ID |
| | | | | GCGCTCACTAC | NO: 70 | GAHY | NO: 146 |
| 4016 | 5.67E−04 | 0.1% | 27 | AACTGGGACGGCCGCG | SEQ ID | NWDGR | SEQ ID |
| | | | | GCATGCACAAC | NO: 71 | GMHN | NO: 147 |
| 4007 | 5.65E−04 | 0.1% | 26 | GACCAGTGGGCAGCAT | SEQ ID | DQWAA | SEQ ID |
| | | | | CGAGAAAGAC | NO: 72 | SRK | NO: 148 |
| 3858 | 5.44E−04 | 0.1% | 27 | AGCATGTGGAGCGACC | SEQ ID | SMWSDP | SEQ ID |
| | | | | CTAGGGCTCAC | NO: 73 | RAH | NO: 149 |
| 3740 | 5.28E−04 | 0.1% | 27 | GAGCCTTGGTGGATCC | SEQ ID | EPWWIQ | SEQ ID |
| | | | | AGCTGGTGGAG | NO: 74 | LVD | NO: 150 |
| 3648 | 5.15E−04 | 0.1% | 27 | ATGTTCAACATCGTGAA | SEQ ID | MFNIVK | SEQ ID |
| | | | | ATGGGCTTAC | NO: 75 | WAY | NO: 151 |
| 3632 | 5.13E−04 | 0.1% | 27 | CCTATGCACATGGTGCT | SEQ ID | PMHMY | SEQ ID |
| | | | | GGAGAACCAG | NO: 76 | LENQ | NO: 152 |

### <Preparation of Fusobacteria for Screening Phage Display Library>

Using a phage display method, M13 bacteriophages having selectivity to Fusobacteria were selected and isolated. A total of 9 types of Fusobacteria were prepared. Among them, Fusobacterium nucleatum subspecies vincentii (PD-B70), Fusobacterium nucleatum subspecies nucleatum (PD-B2298), Fusobacterium nucleatum subspecies animalis (PD-B3771), and Fusobacterium nucleatum subspecies nucleatum (PD-C387) were Fusobacteria derived from patients, and Fusobacterium nucleatum subspecies nucleatum (ATCC-25586), Fusobacterium nucleatum subspecies nucleatum (ATCC-23726), Fusobacterium nucleatum subspecies animalis (ATCC-51191), Fusobacterium nucleatum subspecies polymorphum (ATCC-10953) and Fusobacterium nucleatum subspecies vincentii (ATCC-49256) were Fusobacteria subspecies commercially available from the American Type Culture Collection (ATCC).

The Fusobacteria were cultured in a Gifu Anaerobic Media (GAM) broth in an anaerobic chamber maintained at 37° C. for 18 hours. 10 mL of the cultured broth was centrifuged at 1500×g for 10 minutes to obtain cell pellets, and then resuspended in a solution consisting of 12% [w/v] skim milk powder, 1% [v/v] dimethyl sulfoxide, and 1% [v/v] glycerol. Thereafter, the bacterial suspension was freeze-dried and powdered using a low-temperature freeze dryer, and stored in a 4° C. refrigerator until use.

### <Phage Library Screening for Selecting Peptides Having Selectivity to Fusobacteria>

For library screening, freeze-dried Fusobacteria were dissolved in distilled water (ddH₂O) and then the absorbance was measured at OD600. 9 types of Fusobacteria were mixed in a 1:1 ratio (equal amount), and then the mixture for screening was applied to a plate. Phage display screening was performed using a Trico-C9 peptide library (approximately 8.60×10¹⁰). The library used in the present invention was prepared to express 9 sequence peptides at the end of the gene producing p3, a type of coat protein, in the genome of M13 bacteriophage. First, Fusobacteria and the M13 bacteriophage library were reacted, and unbound bacteriophages were washed and removed 6 times with a phosphate buffer solution containing 0.05% Tween 20. The mixture was eluted with a primary conjugated 0.2 M Glycine-HCl (pH 2.2) solution, and added with Tris-HCl (pH 9.0) for neutralization. The second panning was washed 8 times, the third panning was washed 10 times, and the fourth panning was washed times, and the elution was performed in the same manner as described above. The panning was performed a total of 4 times, and monoclones selective to Fusobacteria were selected in round 3 to round 4. Thereafter, enrichment was performed on single clones showing the greatest positivity. Bacteriophage candidates selective to Fusobacteria were selected through the process, and amino acid sequences were identified through DNA sequencing.

### Example 2. Confirmation of Binding Affinity to Fusobacteria of Bacteriophages Including High-Specific and High-Selective Peptides to Fusobacteria

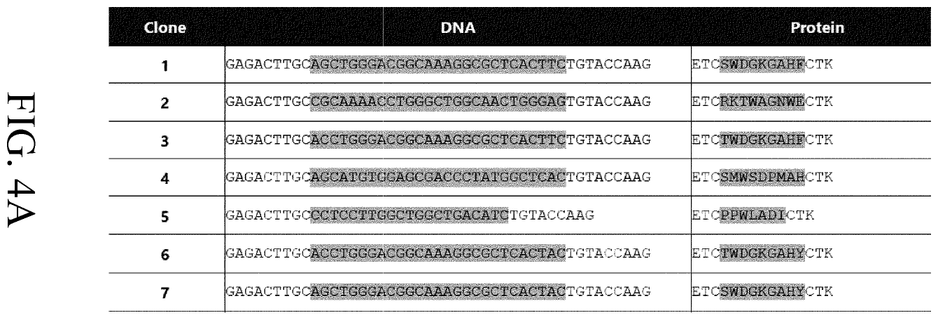

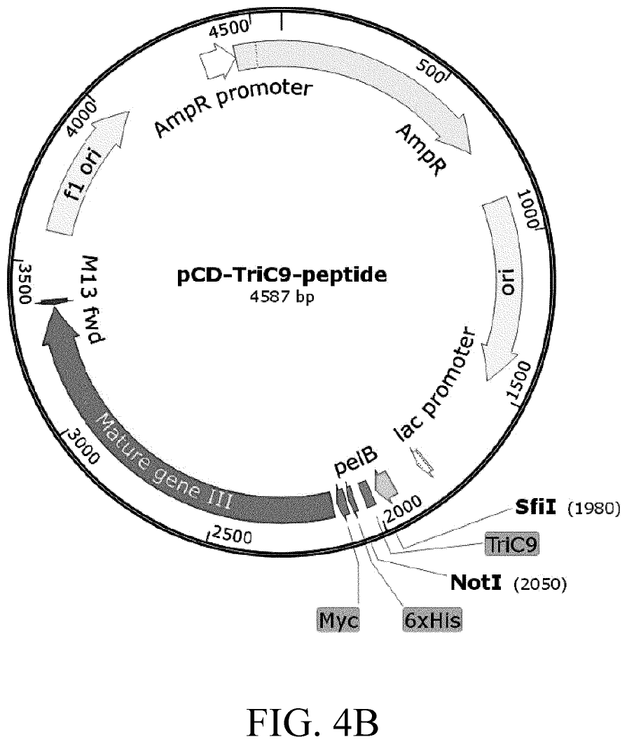

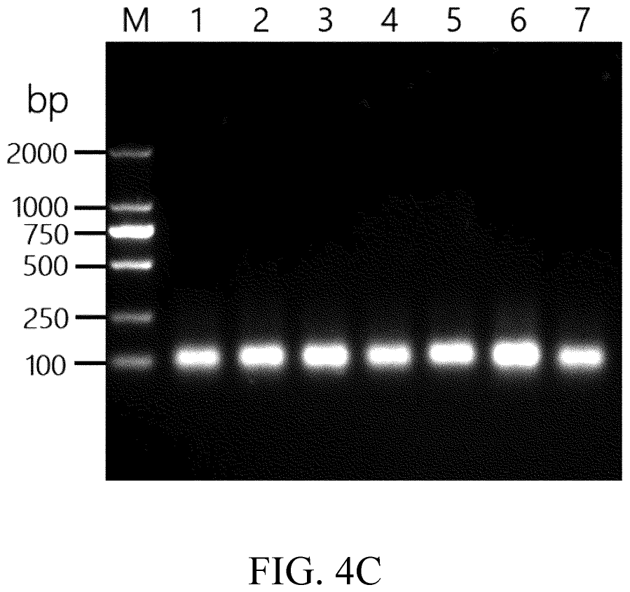

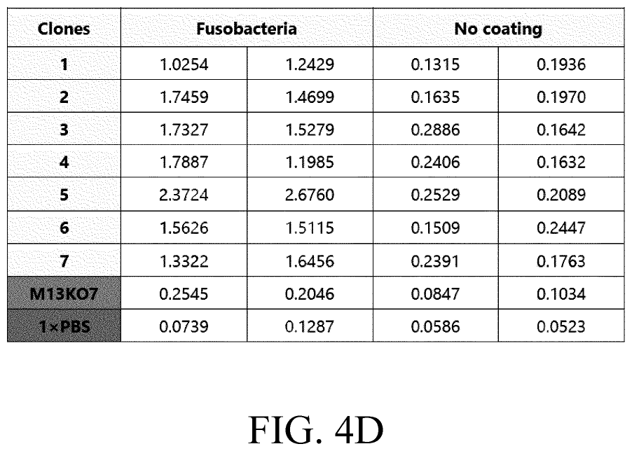

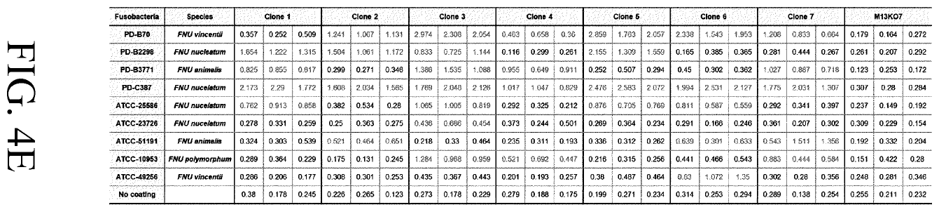

Furthermore, the present inventors intended to verify the bacteriophage peptides derived by the above-described process through ELISA. Accordingly, referring to FIGS. 4A to 4E, there were illustrated results of ELISA verification of the binding affinity and selectivity of the bacteriophage peptides having selectivity to Fusobacteria used in the present invention according to an embodiment of the present invention.

FIG. 4A illustrates sequences of clones 1 to 7 including sequences represented by SEQ ID NOs: 1 to 7 of the bacteriophage peptide for verification of the bacteriophage of the present invention according to an embodiment of the present invention and amino acid sequences translated therefrom.

FIG. 4B illustrates a vector map of pCD-TriC9 phagemid for cloning sequences of clones 1 to 7 containing the sequences represented by SEQ ID NOs: 1 to 7 of the bacteriophage peptide.

FIG. 4C illustrates results of PCR for the sequences represented by SEQ ID NOs: 1 to through cloning of pCD-TriC9 phagemid.

Referring to FIGS. 4A to 4C, for verification of the bacteriophage of the present invention, SfiI and NotI restriction sites were introduced to the 5′ and 3′ ends of the sequences represented by SEQ ID NOs: 1, 2, 3, 4, 5, 6, and 7 to prepare clones 1 to 7 (FIG. 4A). Clones to 7 were introduced into the pCD-TriC9 phagemid of FIG. 4B (FIG. 4B). Furthermore, the cloning results of SEQ ID NOs: 1, 2, 3, 4, 5, 6, and 7 were confirmed by PCR (FIG. 4C).

FIG. 4D illustrates results of measuring the binding affinity to Fusobacteria using ELISA of peptides translated from genes of SEQ ID NOs: 1, 2, 3, 4, 5, 6, and 7 cloned in FIGS. 4A to 4C, that is, peptides of SEQ ID NOs: 77, 78, 79, 80, 81, 82, and 83.

FIG. 4E illustrates ELISA testing results of testing the binding affinity according to 9 types of Fusobacteria of peptides translated from genes of SEQ ID NOs: 1, 2, 3, 4, 5, 6, and 7 cloned in FIGS. 4A to 4C, that is, peptides of SEQ ID NOs: 77, 78, 79, 80, 81, 82, and 83.

Referring to FIG. 4D, it was shown that clones 1 to 7 all had the binding affinity of 1 or more, and among them, SEQ ID NO: 5 (SEQ ID NO: 81) had the highest binding affinity.

Furthermore, referring to FIG. 4E, it was shown that clones 1 to 7 according to 9 types of Fusobacteria had binding affinity to one or more different types of Fusobacteria.

Accordingly, the peptides of SEQ ID NOs: 77 to 83 of the bacteriophage selective to Fusobacteria according to an embodiment of the present invention can target Fusobacteria with high affinity, thereby accurately targeting and removing Fusobacteria, known as harmful bacteria in various diseases, including cancer.

Moreover, the peptides of SEQ ID NOs: 77 to 83 of the bacteriophage selective to Fusobacteria according to an embodiment of the present invention can not only remove Fusobacteria, but also target the Fusobacteria with high affinity as described above, thereby detecting the presence or absence of Fusobacteria with high reliability.

Accordingly, when the bacteriophage peptide having selectivity to Fusobacteria according to an embodiment of the present invention is used as a diagnostic kit, the distribution and presence or absence of metastasis of cancer cells infected with Fusobacteria can be confirmed.

### <Method for Measuring Binding Affinity Using ELISA>

A mixture of 9 types of Fusobacteria or each of the 9 types of Fusobacteria was coated on a plate, and a blocking reaction was performed using a 5% BSA solution to prevent non-specific binding. Each selected M13 bacteriophage was reacted on the plate coated with Fusobacteria. The plate was washed three times with phosphate buffer solution containing 0.1% Tween 20, and then reacted with an M13-HRP antibody. The reaction plate was washed three times with phosphate buffer solution containing 0.1% Tween 20 and reacted using a TMB color solution. The reaction was stopped with 1 M sulfuric acid (H₂SO₄), and the absorbance was measured at OD450 to compare the binding affinity.

### Example 3. Confirmation of Fusobacteria Detection Efficacy Using Bacteriophage Peptides Bound with Fluorescent Labeling Factor

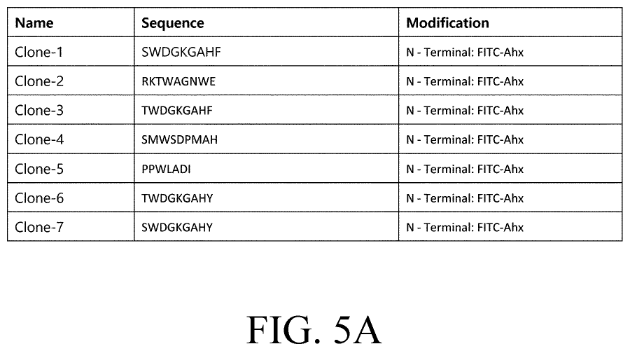

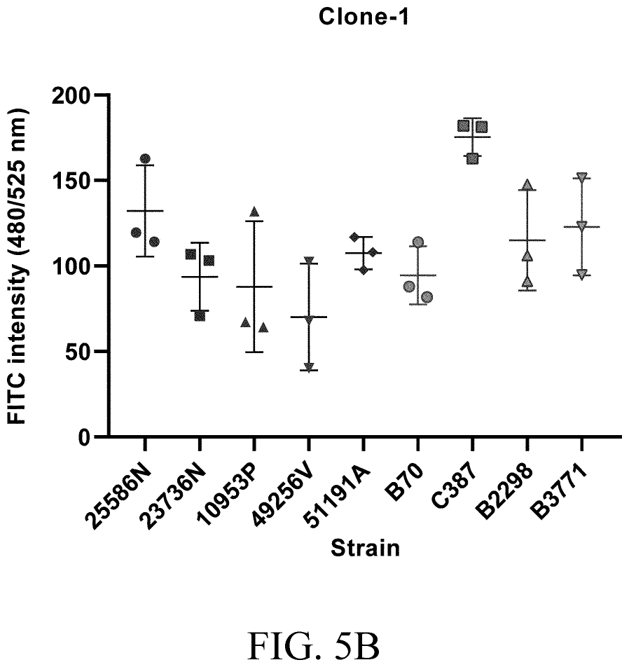

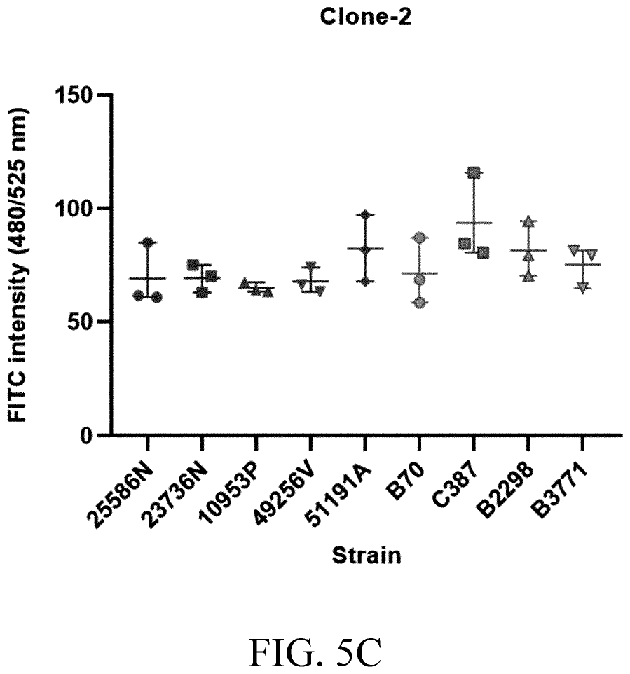

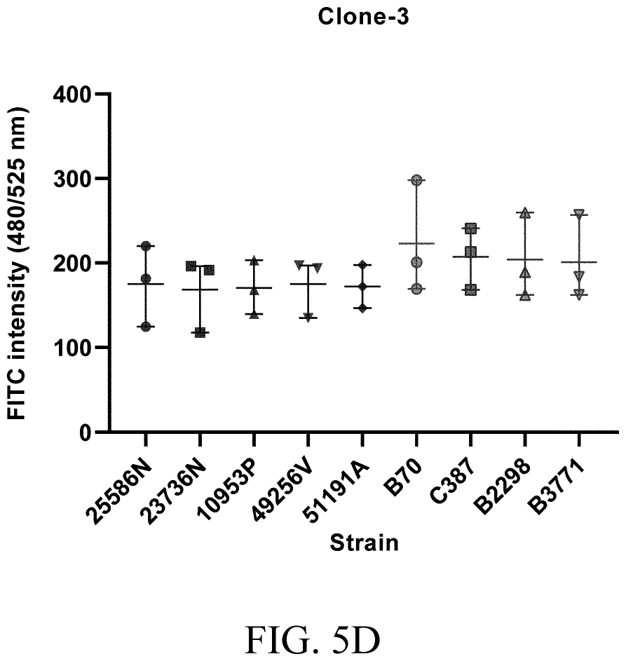

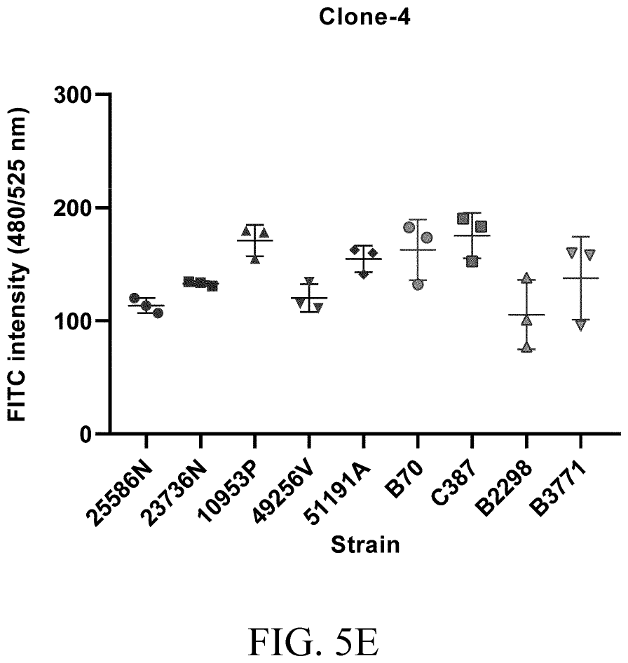

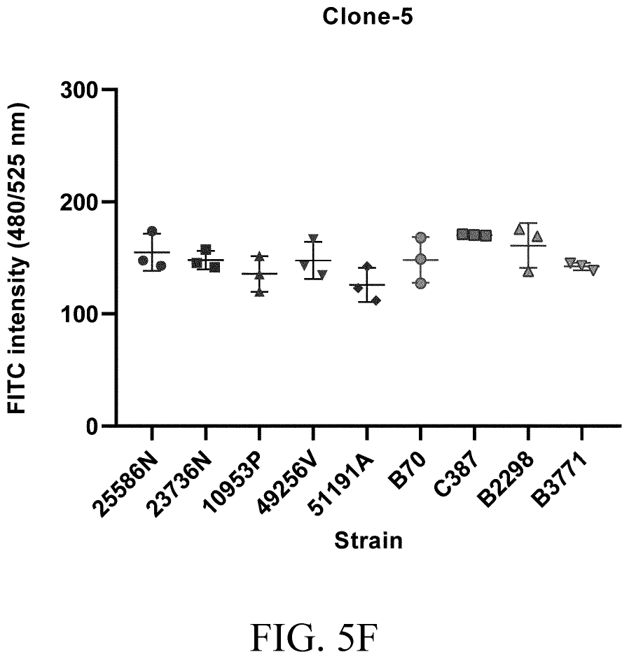

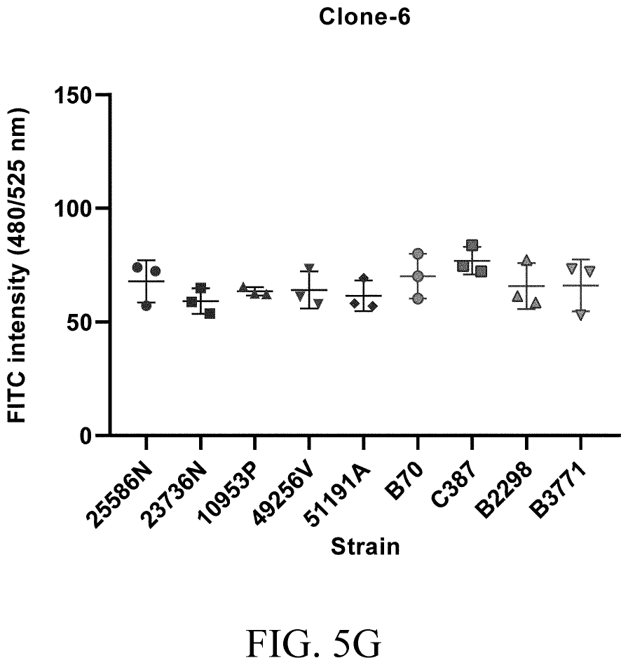

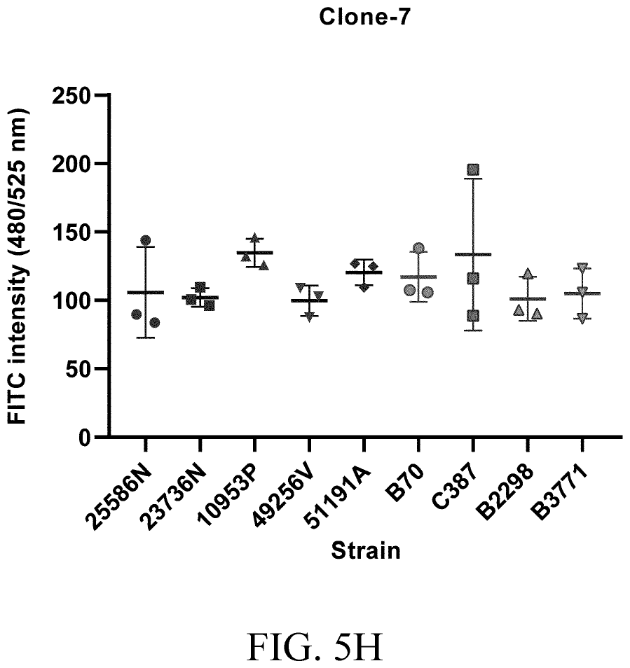

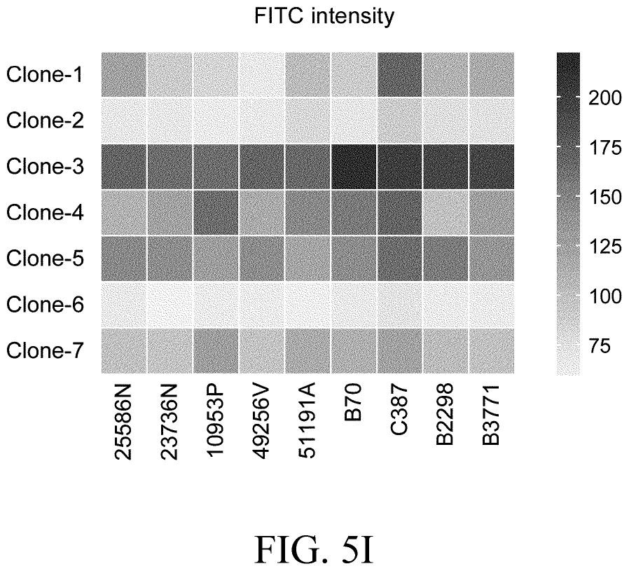

FIGS. 5A to 5I are diagrams illustrating the detection efficacy of bacteria using peptides of SEQ ID NOs: 77 to 83 bound with a fluorescent labeling factor.

Referring to FIG. 5A, 7 peptide sequences represented by SEQ ID NOs: 77 to 83, which were translated from the sequences represented by SEQ ID NOs: 1 to 7, which had high bacterial detection efficacy in Example 1 above, were selected and 7 types of peptides of Clone-1 to Clone-7 bound with fluorescein isothiocyanate (FITC) as a fluorescent labeling factor were synthesized. The synthesis method is not limited, and various known synthesis methods of peptides and substances can be used. The following anticancer agent can be bound with the peptide according to an embodiment of the present invention in the same manner as FITC.

Next, 7 types of peptides of Clone-1 to Clone-7 of FIG. 5A were applied to each well plate coated with 9 types of Fusobacteria, and the amounts of the peptides bound with the Fusobacteria in each well were measured, so that the binding affinity between the peptides of Clone-1 to Clone-7 and 9 types of Fusobacteria was inferred.

In FIGS. 5B to 5H, in an X axis, 25586N refers to ATCC-25586 (Fusobacterium nucleatum subspecies nucleatum), 23726N refers to ATCC-23726 (Fusobacterium nucleatum subspecies nucleatum), 10953P refers to ATCC-10953 (Fusobacterium nucleatum subspecies polymorphum), 49256V refers to ATCC-49256 (Fusobacterium nucleatum subspecies vincentii), 51191A refers to ATCC-51191 (Fusobacterium nucleatum subspecies animalis), B70 refers to PD-B70 (Fusobacterium nucleatum subspecies vincentii), C387 refers to PD-C387 (Fusobacterium nucleatum subspecies nucleatum), B2298 refers to PD-B2298 (Fusobacterium nucleatum subspecies nucleatum), and B3771 refers to PD-B3771 (Fusobacterium nucleatum subspecies animalis).

Referring back to FIGS. 5B to 5H, it was shown that the binding affinity between the peptides of Clone-1 to Clone-7 and 9 types of Fusobacteria was detectable in an FITC fluorescence intensity range of 50 to 250.

FIG. 5I illustrates FITC fluorescence intensity values in FIGS. 5B to 5H by varying shading intensities.

Accordingly, the peptides of SEQ ID NOs: 77 to 83 of the bacteriophage having selectivity to Fusobacteria according to an embodiment of the present invention can be bound with the fluorescent labeling factor to target Fusobacteria and adjacent cancer cells.

Further, the peptides of SEQ ID NOs: 77 to 83 of the bacteriophage having selectivity to Fusobacteria according to an embodiment of the present invention can be bound with various anticancer agents in addition to the fluorescent substances.

In other words, the peptide according to the present invention can be used as an intelligent drug delivery system that selectively delivers the drug to cancer tissues. If the peptide of the present invention is used for cancer treatment by binding to a conventionally known drug, the drug is selectively delivered only to cancer tissues and cancer cells by the peptide of the present invention to increase the efficacy of the drug and significantly reducing the side effects of the drug on normal tissues at the same time.

Accordingly, a chemical anticancer agent can be used as the delivered drug, and the binding to the peptide according to an embodiment of the present invention can be performed in a similar or identical manner to binding to a fluorescent substance such as FITC.

For example, the chemical anticancer agent can be used with at least one of Leucovorin, Levamisole, Irinotecan, Oxaliplatin, Capecitabine, or Uracil/Tegafur, but is not limited thereto, and can include chemical anticancer agents that can be used in cancer in which Fusobacteria is formed, that is, all of Docetaxel, cis-platin, camptothecin, paclitaxel, Tamoxifen, Anasterozole, Gleevec, 5-fluorouracil (5-FU), Floxuridine, Leuprolide, Flutamide, Zoledronate, Doxorubicin, Vincristine, Gemcitabine, Streptozocin, Carboplatin, Topotecan, Belotecan, Vinorelbine, hydroxyurea, nitrosourea, Valrubicin, retinoic acid series, Methotrexate, Mechlorethamine, Chlorambucil, Busulfan, Doxifluridine, Vinblastin, Mitomycin, Prednisone, Testosterone, Mitoxantron, aspirin, salicylates, ibuprofen, naproxen, fenoprofen, indomethacin, phenylbutazone, cyclophosphamide, mechlorethamine, dexamethasone, prednisolone, celecoxib, valdecoxib, nimesulide, daunorubicin, actinomycin-D, etoposide, teniposide, bisantrene, homoharringtonine, busulfan, chlorambucil, melphalan, nitrogen mustard, cortisone, corticosteroid, and the like.

<Method for Measuring Binding Affinity Between Bacteriophage Peptide Bound with Fluorescent Labeling Factor and Fusobacteria>

Fusobacteria were cultured in a Gifu Anaerobic Media (GAM) broth in an anaerobic chamber maintained at 37° C. for 18 hours. The culture solution was centrifuged at 3000×g for 10 minutes to obtain cell pellets, and the cell pellets were washed three times with phosphate buffer solution (PBS). The Fusobacteria were suspended in a carbonate-bicarbonate buffer solution of pH 9.6 and then the absorbance was measured at OD600. A dilution was prepared so that the OD600 value per well was 0.1, and 100 μL was dispensed into a 96-well plate. In order to adsorb the bacteria to the plate, the plate was dried in an incubator at 37° C. Fusobacteria which were not adsorbed to the plate were washed and removed five times with phosphate buffer solution containing 0.05% Tween 20. Thereafter, a blocking reaction to prevent non-specific binding of the peptide was performed using a 1% BSA solution at room temperature for 1 hour. The peptide bound to FITC was prepared so that the final concentration to be treated per well was 100 μM, and after the blocking reaction, the peptide was treated on a plate coated with Fusobacteria and reacted at 4° C. overnight. The plate was washed three times with phosphate buffer solution containing 0.05% Tween 20, and then fluorescence (ex 480 nm/em 525 nm) was measured by ELISA.

### Use of Bacteriophage Peptides Having Selectivity to Fusobacteria According to an Embodiment of the Present Invention

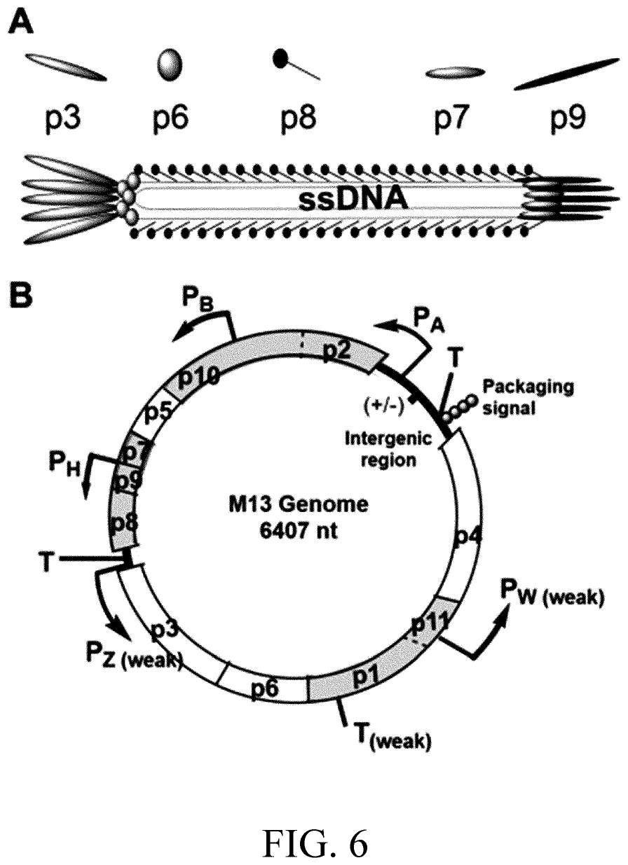

FIGS. 6 to 8 are schematic diagrams for structures of bacteriophages used in the present invention.

First, referring to FIG. 6, the bacteriophage peptide having selectivity to Fusobacteria according to an embodiment of the present invention is displayed in a coat protein P3 region of M13 bacteriophage to specifically recognize (target) Fusobacteria. Meanwhile, P8 and P9, excluding P3, can be cross-linked with various substances.

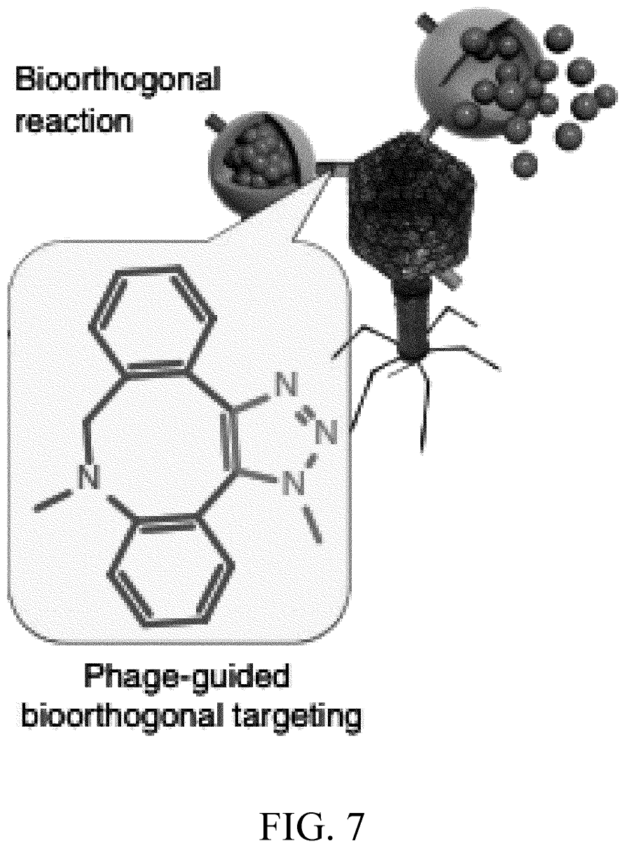

More specifically, referring to FIG. 7, the bacteriophage used in the present invention was used with an amphipathic cross-linker (Phage-guided bioorthogonal targeting) for P8 and P9 to be bound with various compositions. For example, as a composition capable of binding to the P8 and P9 sites, a chemical anticancer agent can be used, and the chemical anticancer agent can be packaged with water-soluble polymers such as dextran and then linked with a cross-linker to bind to the P8 and P9 sites of the bacteriophage. At this time, the chemical anticancer agent can be used with at least one of Leucovorin, Levamisole, Irinotecan, Oxaliplatin, Capecitabine, or Uracil/Tegafur, but is not limited thereto, and can include chemical anticancer agents that can be used in cancer where Fusobacteria is formed, that is, all Docetaxel, cis-platin, camptothecin, paclitaxel, Tamoxifen, Anasterozole, Gleevec, 5-fluorouracil (5-FU), Floxuridine, Leuprolide, Flutamide, Zoledronate, Doxorubicin, Vincristine, Gemcitabine, Streptozocin, Carboplatin, Topotecan, Belotecan, Vinorelbine, hydroxyurea, nitrosourea, Valrubicin, retinoic acid series, Methotrexate, Mechlorethamine, Chlorambucil, Busulfan, Doxifluridine, Vinblastin, Mitomycin, Prednisone, Testosterone, Mitoxantron, aspirin, salicylates, ibuprofen, naproxen, fenoprofen, indomethacin, phenylbutazone, cyclophosphamide, mechlorethamine, dexamethasone, prednisolone, celecoxib, valdecoxib, nimesulide, daunorubicin, actinomycin-D, etoposide, teniposide, bisantrene, homoharringtonine, busulfan, chlorambucil, melphalan, nitrogen mustard, cortisone, corticosteroid, and the like.

Furthermore, a reactive group of the cross-linker can include various reactive groups that can form amide bonds at P8 and P9 of the bacteriophage, including thiol or amine groups.

Furthermore, the bacteriophage can be reached intracellularly. Accordingly, when the bacteriophage including the bacteriophage peptide having selectivity to Fusobacteria according to an embodiment of the present invention further includes the above-described chemical anticancer agent, it is possible to invade the inside of cancer cells, thereby further improving drug delivery efficiency and also improving the effect of anticancer treatment.

In addition, the bacteriophage including the bacteriophage peptide having selectivity to Fusobacteria according to an embodiment of the present invention can further include a genetic drug as well as the chemical anticancer agent. For example, the gene drug can be small interfering RNA (siRNA) and single guide RNA (sgRNA) targeting at least one protein selected from the group consisting of Bcl-2 Antagonist X (Bax), B-cell lymphoma 2 (BCl-2), Focal adhesion kinase, Matrix metalloproteinase, Vascular endothelial growth factor (VEGF), Fatty acid synthase, Multiple drug resistance protein (MDR), Harvey rat sarcoma viral oncogene homolog (H-Ras), Kirsten-rat sarcoma viral oncogene homolog (K-Ras), Polo Like Kinase 1 (PLK-1), Transforming growth factor beta (TGF-β), Signal Transducer And Activator Of Transcription 3 (STAT3), Epidermal growth factor receptor (EGFR), Protein kinase C alpha (PKC-α), Epstein-Barr virus, human papillomavirus E6 (HPV E6), BCR-ABL fusion gene, and Telomerase of cancer cells, but is not limited thereto. Accordingly, the bacteriophage including the bacteriophage peptide having selectivity to Fusobacteria according to an embodiment of the present invention delivers the genetic drug into cancer cells, thereby modifying the genes of cancer cells and resulting in an anti-cancer effect.

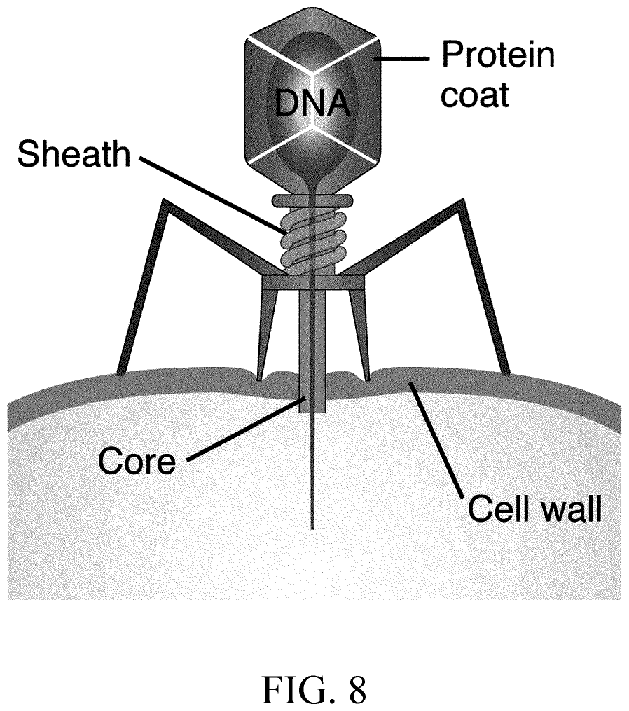

In addition, the bacteriophage including the bacteriophage peptide having selectivity to Fusobacteria according to an embodiment of the present invention can be applied with genetic scissors (CRISPR-Cas9). For example, referring to FIG. 8, the bacteriophage including the bacteriophage peptide having selectivity to Fusobacteria according to an embodiment of the present invention can be introduced with the genetic scissors, and DNA of the bacteriophage can include a DNA sequence that has the ability to kill cancer cells. Accordingly, when the bacteriophage is injected, the DNA sequence having the ability to kill cancer cells is injected into Fusobacteria or various cancer cells in which Fusobacteria exist, and thus its protein can be displayed.

Accordingly, the bacteriophage including the bacteriophage peptide having selectivity to Fusobacteria according to an embodiment of the present invention can have anti-cancer effects by causing direct genetic modification in cancer cells.

Although the exemplary embodiments of the present invention have been described in detail with reference to the accompanying drawings, the present invention is not limited thereto and can be embodied in many different forms without departing from the technical concept of the present invention. Therefore, the exemplary embodiments of the present invention are provided for illustrative purposes only but not intended to limit the technical concept of the present invention. The scope of the technical concept of the present invention is not limited thereto. Therefore, it should be appreciated that the aforementioned exemplary embodiments are illustrative in all aspects and are not restricted. The protective scope of the present invention should be construed on the basis of the appended claims, and all the technical ideas in the equivalent scope thereof should be construed as falling within the scope of the present invention.

### [National Research and Development Project Supporting the Invention]

[Project Unique Number] 1711118087
[Project Number] 2020R1F1A1066973
[Department Name] Ministry of Science and ICT
[Project Management (Special) Institution Name] National Research Foundation of Korea
[Research Project Name] Personal basic research (Ministry of Science and ICT) (R&D)
[Research Subject Name] Study on effect of Fusobacteria infection on immune molecular profiling in colon cancer tissue tumor microenvironment
[Contribution ratio] 34/100
[Project performance institute name] Yonsei University
[Research Period] 20200601 to 20210228

### [National Research and Development Project Supporting the Invention]

[Project Unique Number] 1711114441
[Project Number] 2019R1C1C1006709
[Department Name] Ministry of Science and ICT
[Project Management (Special) Institution Name] National Research Foundation of Korea
[Research Project Name] Personal basic research (Ministry of Science and ICT) (R&D)
[Research Subject Name] Characterization of methylation and epigenetic modification of DNA from tumor-derived exosomes and its clinical application as cancer biomarker
[Contribution ratio] 33/100
[Project performance institute name] Yonsei University
[Research Period] 20200301 to 20210228

### [National Research and Development Project Supporting the Invention]

[Project Unique Number] 1345323642
[Project Number] 2019R1A6A3A01096180
[Department Name] Ministry of Education
[Project Management (Special) Institution Name] National Research Foundation of Korea
[Research Project Name] Establishment of Research Foundation for Science and Engineering (R&D)
[Research Subject Name] Development and clinical availability of treatment strategies for targeting colorectal cancer associated microbiota using bacteriophages and CRISPR/Cas9 technology
[Contribution ratio] 33/100
[Project performance institute name] Yonsei University
[Research Period] 20200901 to 20210831

1. A peptide targeting Fusobacteria comprising a peptide consisting of an amino acid sequence selected from the group consisting of SEQ ID NO: 77 to SEQ ID NO: 83, wherein the peptide targets Fusobacteria.

2. The peptide targeting Fusobacteria of claim 1, wherein the peptide is displayed in a major coat protein P3 of a M13 bacteriophage.

3. An expression vector comprising one of sequences represented by SEQ ID NOs: 1 to 7 encoding an amino acid sequence selected from the group consisting of SEQ ID NOs: 77 to 83 according to claim 1.

4. The expression vector of claim 3, wherein the expression vector is a phagemid vector.

5. A transformant transformed with the expression vector according to claim 3.

6. A composition for diagnosing cancer, including a peptide targeting Fusobacteria consisting of an amino acid sequence selected from the group consisting of SEQ ID NOs: 77 to 83.

7. The composition for diagnosing cancer of claim 6, wherein the peptide targeting Fusobacteria is labeled with one selected from the group consisting of a color enzyme, a radioisotope, a chromophore, a luminescent substance, a fluorescer, super paramagnetic particles, and ultrasuper paramagnetic particles.

8. The composition for diagnosing cancer of claim 6, wherein the peptide targeting Fusobacteria targets and diagnoses at least one cancer cell of colorectal cancer, melanoma, pancreatic cancer, liver cancer, stomach cancer, colon cancer, lung cancer, ovarian cancer, breast cancer, and cervical cancer, infected with Fusobacteria.

9. A drug delivery composition comprising a peptide targeting Fusobacteria consisting of an amino acid sequence selected from the group consisting of SEQ ID NOs: 77 to 83.

10. The drug delivery composition of claim 9, wherein the drug is physically or chemically bound to the peptide targeting Fusobacteria, and is an anticancer agent for treating cancer cells infected with Fusobacteria.

11. The drug delivery composition of claim 10, wherein the anticancer agent targets and treats at least one cancer cell of colorectal cancer, melanoma, pancreatic cancer, liver cancer, stomach cancer, colon cancer, lung cancer, ovarian cancer, breast cancer, and cervical cancer, infected with Fusobacteria.

12. The drug delivery composition of claim 10, wherein the anticancer agent is selected from the group consisting of Leucovorin, Levamisole, Irinotecan, Oxaliplatin, Capecitabine, Uracil/Tegafur, Docetaxel, cis-platin, camptothecin, paclitaxel, Tamoxifen, Anasterozole, Gleevec, 5-fluorouracil (5-FU), Floxuridine, Leuprolide, Flutamide, Zoledronate, Doxorubicin, Vincristine, Gemcitabine, Streptozocin, Carboplatin, Topotecan, Belotecan, Vinorelbine, hydroxyurea, nitrosourea, Valrubicin, retinoic acid series, Methotrexate, Mechlorethamine, Chlorambucil, Busulfan, Doxifluridine, Vinblastin, Mitomycin, Prednisone, Testosterone, Mitoxantron, aspirin, salicylates, ibuprofen, naproxen, fenoprofen, indomethacin, phenylbutazone, cyclophosphamide, mechlorethamine, dexamethasone, prednisolone, celecoxib, valdecoxib, nimesulide, daunorubicin, actinomycin-D, etoposide, teniposide, bisantrene, homoharringtonine, busulfan, chlorambucil, melphalan, nitrogen mustard, cortisone, and corticosteroid.

13. The drug delivery composition of claim 9, wherein the anticancer agent is physically or chemically bound to the peptide targeting Fusobacteria, and is a gene drug for treating cancer cells infected with Fusobacteria.

14. The drug delivery composition of claim 13, wherein the gene drug is selected from the group consisting of small interfering RNA (siRNA) and single guide RNA (sgRNA) targeting at least one protein selected from the group consisting of Bcl-2 Antagonist X (Bax), B-cell lymphoma 2 (BCl-2), Focal adhesion kinase, Matrix metalloproteinase, Vascular endothelial growth factor (VEGF), Fatty acid synthase, Multiple drug resistance protein (MDR), Harvey rat sarcoma viral oncogene homolog (H-Ras), Kirsten-rat sarcoma viral oncogene homolog (K-Ras), Polo Like Kinase 1 (PLK-1), Transforming growth factor beta (TGF-β), Signal Transducer And Activator Of Transcription 3 (STAT3), Epidermal growth factor receptor (EGFR), Protein kinase C alpha (PKC-α), Epstein-Barr virus, human papillomavirus E6 (HPV E6), BCR-ABL fusion gene, and Telomerase of cancer cells.
